(19) United States
(12) Patent Application Publication
(10) Pub. No.: US 2014/0120167 A1
(43) Pub. Date: May 1, 2014

(54) MULTIFUNCTIONAL CHEMO- AND MECHANICAL THERAPEUTICS

(71) Applicant: WILLIAM MARSH RICE UNIVERSITY, HOUSTON, TX (US)

(72) Inventors: Dmitri Lapotko, Pearland, TX (US); Katsiaryna Hleb, Houston, TX (US); Michael E. Kupferman, Houston, TX (US); Daniel Scudder Wagner, Houston, TX (US); Vladimir Torchilin, Charlestown, MA (US); Xiangwei Wu, Houston, TX (US)

(73) Assignee: WILLIAM MARSH RICE UNIVERSITY, HOUSTON, TX (US)

(21) Appl. No.: 14/067,570

(22) Filed: Oct. 30, 2013

Related U.S. Application Data
(60) Provisional application No. 61/720,135, filed on Oct. 30, 2012.

(51) Int. Cl. A61K 41/00; C12N 13/00; G01N 33/483; A61K 9/51

(52) U.S. Cl. CPC A61K 41/00; A61K 9/51; C12N 13/00; G01N 33/4833
USPC 424/490; 514/769; 435/173.1; 435/29; 604/20; 600/2

(57) ABSTRACT

Methods of treating diseases through the intracellular enhancement and synergy of chemo- and radiation-therapies by employing cancer cell-specific on-demand mechanical intracellular impact. The methods, quadrapeutics, combines four clinically validated modalities: encapsulated drugs, colloidal gold nanoparticles (GNPs), near-infrared short laser pulses, and X-rays.

### CROSS-REFERENCE TO RELATED APPLICATIONS

This Application claims the benefit of U.S. Provisional Application Ser. No. 61/720,135 filed on Oct. 30, 2012, which is incorporated by reference.

### STATEMENT OF GOVERNMENT INTEREST

This invention was made with government support under Grant Numbers R01GM094816, R01CA128486, 5U54151881-012, and S10RR026399-01, awarded by the National Institutes of Health. The government has certain rights in the invention.

### BACKGROUND

Present day chemo- and radio-therapies are associated with severe side effects caused by the drug-and radiation-induced action on healthy tissues. In addition, disease-specific (for example, cancer) cells often become resistant to an active chemotherapeutic agent or multiple resistant to a plethora of chemotherapeutic agents and to radiation.

Existing methods use gold nanoparticles for the localized enhancement of radiation or as drug carriers (or as components of the complex drug nanocarriers) together with radiation. Previous art has employed the release of encapsulated drug with optically activated plasmonic nanoparticles (including the generation of plasmonic (or vapor) nanobubbles) and the delivery of such drug into target cells, but in all existing methods, plasmonic nanoparticles are considered as a component of the nano-complex that delivers the drug. In other words, the capsule with the molecular agent in current methods requires the incorporation of the source of thermal or mechanical impact (required to release the drug) such as gold nanoparticles. This has been done because the drug release mechanism requires co-localization of the capsule and plasmonic nanoparticle. However, these methods show only incremental gains in therapeutic efficacy, require high doses of nanoparticles and drugs, and do not prevent their non-specific uptake by normal cells. Therefore, previous approaches cannot provide intracellular co-localized and synchronized enhancement of drug concentration and radiation intensity specifically in cancer cells.

Accordingly, there is a need to improve the efficacy and selectivity of existing chemotherapy and radiotherapy when both are applied to treat a disease, and to reduce non-specific toxicity and duration of the two above treatments.

The use of surgery and chemo- and chemoradiation therapies against cancers occurring in vitally important anatomic locations such as the head and neck (as well as the brain, prostate and lungs) presents several limitations: (1) tumors that are not completely resected result in microscopic residual disease (MRD); (2) the resection of tumors that are intertwined with functionally or cosmetically important organs causes functional and cosmetic damage; (3) residual cancer cells often develop a high resistance to chemo- and radio-therapy, thus resulting in high levels of local regional recurrence; and (4) high doses of drugs and X-rays induce severe non-specific toxicities. These limitations ultimately compromise patients' survival rates (which for head and neck cancers have remained relatively unchanged for the past 30 years) and profoundly impact patient quality of life, cosmesis and psychological health. Therefore, developing a novel approach that (i) selectively detects and rapidly eliminates resistant residual cancer cells and tumors, (ii) preserves the functionality of co-localized normal tissues and (iii) reduces non-specific toxicity and treatment time, is highly desirable.

### DRAWINGS

The patent or application file contains at least one drawing executed in color. Copies of this patent or patent application publication with color drawing(s) will be provided by the Office upon request and payment of the necessary fee.

Some specific example embodiments of the disclosure may be understood by referring, in part, to the following description and the accompanying drawings.

FIG. 1 shows principle of the plasmonic enhancement of the therapeutic efficacy and selectivity of drugs and radiation. (a): The large mixed intracellular clusters of separately targeted gold (plasmonic) nanoparticles (GNPs) and drug nanocarriers are self-assembled by cancer cells (top), but not by normal cells (bottom). (b): The localized intracellular release of the drug (green dots) due to the cancer-cell specific generation of a plasmonic nanobubble (PNB) that disrupts the nanocarrier and endosome and locally ejects the drug. In normal cells non-specific uptake of fewer GNPs is insufficient to generate PNBs and no release is triggered. (c): The intracellular amplification of the external X-rays by a cluster of GNPs is co-localized with the locally released molecular payload to maximally enhance the therapeutic effect in cancer cells but not in normal cells.

FIG. 2 shows PNB-enhanced intracellular on-demand release of encapsulated Calcein Green dye. Confocal microscopy images of living cells treated with green fluorescent dye-loaded liposomes conjugated with 2C5 antibody (detected as green in fluorescent mode) and gold NP-C225 conjugates (detected as red in scattering mode). (a): Before the laser pulse (green fluorescence is quenched and dimmed in intact liposomes). (b): During the laser pulse: clusters of gold NPs (I) following exposure to a single laser pulse (70 ps, 532 nm, 40 mJ cm⁻²) generate co-localized PNBs (II, optical scattering time-resolved image) that are quantified through their optical scattering time-response (b, III). (c): Immediately after a single laser pulse. (d): Cell population-averaged kinetics of image pixel amplitude of green fluorescence (solid) and size of fluorescent zone (hollow) (n=150). Data: mean±s.d. Calcein Green excitation/emission/bandpass wavelength: 488/530/25 nm.

FIG. 3 shows cellular specificity of PNB-enhanced intracellular release. (a): Merged bright field and confocal tri-color fluorescent image of co-culture of EGFR-positive (red) and EGFR-negative (blue) cells treated with conjugates of GNPs and liposomal green dye (not seen due to the fluorescence quenching in intact liposomes); (b): Tri-color fluorescent image of the same field as in (a), dashed contours show cellular boundary; (c): Optical scattering time-responses show the selective generation of PNBs only in red EGFR-positive cancer cells following simultaneous exposure of all cells to a single laser pulse (70 ps, 532 nm, 40 mJ cm⁻²); (d): Cells at 5-10 min after exposure to a single laser pulse show the localized release of green dye only in EGFR-positive cell due to the selective generation of small PNB that disrupted the dye nanocarrier and endosome and locally ejected the dye (green). Non-specific uptake of gold NPs in EGFR-negative cell did not generate PNBs and thus did not trigger the dye release; (e) Tri-color fluorescent image of the same field as in (d), dashed contours show cellular boundary; and (f) Cell population-averaged image pixel amplitudes of co-culture of EGFR-positive (target; red bars) and EGFR-negative (non-target; blue bars) cells before and after their exposure to a single laser pulse as specified above (green bars: Calcein Green fluorescence), sample size: n=180. Data: mean±s.d. Calcein Green excitation/emission/bandpass wavelength: 488/530/25 nm; Calcein Red-Orange excitation/emission/bandpass wavelength: 543/574/26 nm; DAPI excitation/emission/bandpass wavelength: 405/462/44 nm.

FIG. 4 shows surviving fraction (clonogenicity) of HN31 cells (red circles) and NOM9 cells (black circles) as a function of (a) drug concentration (Doxil: solid; Paclitaxel: hollow), (b) laser pulse fluence; (c) X-ray dose. Vertical lines show the doses for zero clonogenicity (SF=0): long dash—Doxil, short dash—Paclitaxel; (d) Surviving fraction of HN31 cells as a function of the treatment mode (I—intact cells; PNB—GNP-C225 conjugates and laser pulse (70 ps, 780 nm, 45 mJ cm⁻²); XR—X-rays, 4 Gy; GNP+XR—GNP-C225 and X-rays; D+XR drug (grey—Doxil, 2 μg mL⁻¹; red—Paclitaxel, 33 ng mL⁻¹) and X-rays; PNB+XR1—GNP-C225, laser pulse and X-rays 1 hour after laser exposure; D+PNB—GNP-C225, drug and laser pulse; D+PNB+XR1—GNP-C225, drug, laser pulse and X-rays 1 hour after laser exposure; D+PNB+XR6—GNP-C225, drug, laser pulse and X-rays 6 hours after laser exposure), black arrows indicate a zero level of clonogenicity; (e) Surviving fraction of NOM9 as a function of the treatment mode identical to that in (d); (f) Comparison of clonogenicity of cancer HN31 (solid) and normal (NOM9) (hollow) cells treated with standard chemoradiation therapy and quadrapeutics with Doxil (Dox) and micellar Paclitaxel (Ptx), black arrows indicate zero level of clonogenicity. Data: mean±s.d. for the three independent experiments, *p<0.05, **p>0.05.

FIG. 5 shows a quadrapeutic treatment of HNSCC in murine models. (a-c) In vitro pre-treated HN31 cells injected into mice: (a, b) images of the animals 15 days after the cell pre-treatment and injection: intact HN31 cells (I), chemoradiation therapy-treated cells (S), PNB-treated cells (L) and quadrapeutics-treated (Q) cells (Doxil, 2 μg mL⁻¹, GNP-C225, 2.4×1010 particles mL⁻¹, laser pulse, 780 nm, 45 mJ cm⁻², X-rays, 4 Gy); (c) tumor incidence rate (shaded bar) and volumes (solid bar) in three treatment groups measured 15 days after the cell pre-treatment and injection. Data: mean±s.e.m. for independent experiments (I: n=5, S: n=6, Q: n=4); and (d-g) Primary tumor model: Bioluminescent images of the animal before, (d), and one week after, (e), single-time treatments with quadrapeutics (left flank, Doxil-C225, 1 mg kg⁻¹ and GNP-C225, 0.8 mg kg⁻¹, both i.v. injected, laser pulse, 780 nm, 45 mJ cm⁻², local and X-rays, 4 Gy) and chemoradiation (right flank, the same doses of Doxil and X-rays), (f) Tumor volumes one week after the treatment (I—untreated, S-chemoradiation, Q-quadrapeutics), (g) Time course of the primary tumor volumes after the single-time in vivo administration of the following treatments against primary HNSCC tumor in xenograft murine model: quadrapeutics (Doxil-C225, 1 mg kg⁻¹, GNP-C225, 0.8 mg kg⁻¹, laser pulse, 780 nm, 45 mJ cm⁻², X-rays, 4 Gy,)—red: tumor volume of quadrapeutic-treated animals (n=11); chemoradiation (identical to the above drug and X-ray dose)—blue: tumor volume of chemoradiation-treated animals (n=11); PNB alone (identical to the above GNP and laser doses)—orange: of the PNB-treated animals (n=4); the tumor volume of untreated animals (n=6)—black. Data: mean±s.e.m.

FIG. 6 shows an evaluation of quadrapeutics for intra-operative real-time diagnosis and treatment of HNSCC MRD (a) Experimental model of intra-operative diagnosis and treatment of MRD: primary tumor (tumor), residual micro-tumor (RT) in surgical margins (SM), laser beam scan range and acoustic sensor (AS), and its PNB-specific acoustic response (inset). (b) Intra-operative diagnosis of MRD: Amplitudes of the PNB acoustic responses obtained during the laser scans in GNP-treated and untreated animals for primary tumors before their resection (T), surgical margins immediately after the tumor resection (SM), and adjacent normal tissue (N); horizontal grey line shows the background signal level. Data: mean±s.e.m. (c-e) Intra-operative treatment of MRD: fluorescent images of GFP-encoded tumors obtained 28 days after (c) surgery alone (I), (d) surgery and adjuvant chemoradiation (S), (e) surgery and adjuvant quadrapeutics (all doses identical to a primary model as shown above), (f): Metrics of recurrent tumors obtained in 28 days after the intra-operative treatment of MRD: solid bar—level of fluorescence in GFP-encoded HNSCC cells; shaded bar—incidence rate of a tumor (I: n=5; S: n=6, Q: n=7). Data: mean±s.e.m. *p<0.05, **p>0.05.

FIG. 7A shows viability of target (HN31) cells measured 72 h after applying paclitaxel-loaded 14 nm micelles (M), gold nanoparticles, PNBs and x-rays (M+NP—gold conjugates and paclitaxel loaded micelles conjugated to C225, M+PNB—laser pulse was applied to gold NP and micelle-treated cells). FIG. 7B shows dependence of the viability of cancer cells upon the external dose of the radiation.

FIG. 8 shows the complex viability of cancer (HN31, solid red) and normal (NOM9, solid green) cells measured 72 h after applying specific treatments. Blue bars show the PNB lifetime in cancer (blue solid) and normal (blue hollow) cells. The treatment modes: I: intact cells; GNP: cells treated by gold 60 nm spheres conjugated with C225; GNP+Dox: cells treated with GNP and soluble incapsulated drug doxorubicin (Doxil), 5 μg mL⁻¹, conjugated with C225; PNB: single laser pulse applied to GNP-C225-treated cells; Dox+PNB: single laser pulse was applied to GNP-C225- and Doxil-C225-treated cells. GNP+Ptx: GNP and incapsulated poorly soluble drug paclitaxel (Ptx), 0.065 μg/mL, conjugated with C225; Ptx+PNB: single laser pulse was applied to GNP-C225- and Ptx-C225-treated cells. Laser treatment was a single pulse, 70 ps, 532 nm, 40 mJ cm⁻². *p<0.05, **p>0.05.

FIG. 9 shows western blot analysis of HN31 squamous carcinoma and immortalized normal human oral kerotinocyte NOM9 cells for the expression level of epidermal growth factor receptor (EGFR).

FIG. 10 shows fluorescence image pixel amplitude of intact (black) and alcohol dissolved (hollow) liposomes. Calcein Green excitation/emission/bandpass wavelength: 488/530/25 nm.

FIG. 11 shows the confocal images of HN31 cells treated with GNP-C225 conjugates and Calcein-Green loaded liposomes conjugated with 2C5 antibody and exposed to a single laser pulse (70 ps, 532 nm, 40 mJ cm⁻²). Top: merged bright field and fluorescence images of the cells at different distances. Bottom: the fluorescence images of the same cells. The images were selected from the Z-stack obtained by using a LSM710 laser confocal microscope. Calcein Green excitation/emission/bandpass wavelength: 488/530/25 nm.

FIG. 12(a) shows averaged size of GNP clusters in cancer (HN31) and normal (NOM9) cells (measured in vitro as the GNP scattering pixel image amplitude using a laser confocal microscope); and FIG. 12(b) shows PNB generation threshold fluence of the excitation laser pulse as a function of GNP cluster size (measured through scattering pixel amplitude of GNP cluster image in individual cells).

FIG. 13(a) shows dependence of PNB lifetime versus the laser pulse fluence of a 70 ps laser pulse in cancer (HN31, red) and normal (NOM9, black) cells treated with GNP-C225 conjugates; and FIG. 13(b) shows PNB lifetime spectra in individual cancer cells in vitro targeted with GNP-C225 conjugates.

FIG. 14 shows the complex viability of cancer HN31 measured 72 h after applying specific treatments. (a): PNB: single laser pulse applied to GNP-C225-treated cells; Dox: cells treated with plain doxorubicin-loaded liposomes; Dox+PNB: single laser pulse applied to GNP-C225- and plain doxorubicin-loaded liposomes-treated cells; Dox-C225: cells treated with conjugated doxorubicin-loaded liposomes; Dox-C225+PNB: single laser pulse applied to GNP-C225- and Dox-C225-treated cells. (b) Ptx: plain paclitaxel-loaded micelles-treated cells; Ptx+PNB: single laser pulse applied to GNP-C225- and plain paclitaxel-loaded micelles-treated cells; Ptx-mAb: cells treated with conjugated paclitaxel-loaded micelles; Ptx-mAb+PNB: single laser pulse applied to GNP-C225- and conjugated (C225) paclitaxel-loaded micelles-treated cells. The effect of dual targeting with Ptx-2C5 and GNP-C225 (black, the above conjugates are shown as Ptx-mAb) (*p<0.05, **p>0.05).

FIG. 15 shows the complex viability of cancer (HN31) and normal (NOM9) cells measured 72 h after applying specific treatments. (a) Effect of a single X-ray dose (10 Gy) applied within 30 min after treatment to cancer cells pre-treated as in (FIG. 8b) (red—without X-rays, drug dose reduced to 0.05 μg mL⁻¹; purple—with X-rays). (b) The effect of a single X-ray dose (10 Gy) on cancer (purple) and normal (green) cells pre-treated as in (FIG. 8b) under the reduced concentration of Ptx (0.05 μg mL⁻¹). Blue bars show the PNB lifetime in cancer (solid) and normal (hollow) cells. Laser treatment was a single pulse, 70 ps, 532 nm, 40 mJ cm⁻². *p<0.05, **p>0.05.

FIG. 16 shows transmission electron microscopy images of solid 60 nm GNP-C225 conjugates in tumor (a) and adjacent muscle tissue (b) 24 h after systemic injection of GNP-C225 into the mouse; (c) average size of GNP clusters in tumor and adjacent tissue (according to TEM images).

FIG. 17(a) shows acoustic responses to a single laser pulse from a primary tumor (red) and adjacent normal tissue (black) in a mouse systemically treated with GNP-C225 conjugates; FIG. 17(b) shows amplitude of the PNB acoustic response as function of the laser pulse fluence in tumor (red) and normal tissue (black) in vivo in a mouse systemically treated with GNP-C225 conjugates; FIG. 17(c) shows spectra of acoustic responses of a tumor (red) and intact tissue (black) after systemic delivery of GNP-C225 conjugates in a mouse. Acoustic responses were obtained 24 hours after the systemic GNP-C225 injection; and FIG. 17(d) shows scans of GNP-treated animal: PNB signal amplitudes for primary tumor (solid green), surgical margins after tumor resection (solid red) and primary tumor in intact animal that was not treated with GNPs (solid black), standard photoacoustic small imaging system (Vevo LAZR, Visual Sonics) signals for the same animals (dashed lines).

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as defined by the appended claims.

### DESCRIPTION

The present disclosure generally relates to treatments of diseases, and more particularly to systems and methods related to chemotherapy and radiotherapy applied to treat a disease, such as, for example, cancer.

A major drawback of existing chemotherapy and radiotherapy techniques is their non-specific toxicity and duration. That is, it is desirable to make chemotherapy and radiotherapy techniques targeted more specifically at disease-specific cells while sparing normal cells and organs. More specifically, it is desirable to transform current macro-practices into a cell level on-demand therapy with high efficacy even against resistant tumors and cancer cell-specificity that can be applied after standard treatments fail.

The systems and methods of the present disclosure, in some embodiments, improve the efficacy and selectivity of existing chemotherapy and radiotherapy when both are applied to treat the same disease, and to reduce non-specific toxicity and duration of the two above treatments. These systems and methods, in some embodiments, will further add at least the following benefits, among others: (1) Current protocols for chemo- and radio-therapies may be employed with standard drugs and radiation but under significantly reduced doses of both; (2) Two additional safe components may be added to disease treatment in some embodiments—near-infrared low energy pulsed laser radiation and colloidal gold (both of which are FDA-compatible); (3) In some embodiments, treatment time may be reduced to a single procedure. As a result, the survival rate of patients suffering from disease, such as cancer patients, will increase while the treatment burden will decrease, thereby also improving patient quality of life.

Furthermore, the systems and methods of some embodiments of the present disclosure can be relatively quickly translated into clinical use because these systems and methods rely on novel applications, combinations, and/or modifications of existing modalities that result in reduced doses and treatment times. For example, one impact of several embodiments of the present disclosure is in the novel downscaling of two conventional treatments to the cell level while providing high speed of drug delivery and high selectivity of the treatment. The methods of such embodiments change the current paradigm of whole body systemic treatment to cell level treatment. For example, standard chemo- and radio-therapies may, through the use of the systems and methods of some embodiments, be down-scaled to individual cancer cells while sparing normal cells. This will improve the survival rate, will reduce treatment time and will minimize adverse effects of cancer treatment.

In certain embodiments, the present invention includes an intracellular enhancement and synergy of chemo- and radiation-therapies by employing cancer cell-specific on-demand mechanical intracellular impact. In some embodiments, a novel method, termed quadrapeutics, includes four clinically validated modalities: encapsulated drugs, colloidal gold nanoparticles (GNPs), near-infrared short laser pulses, and X-rays.

The present disclosure provides, according to certain embodiments, methods comprising: introducing into a cell at least one gold nanoparticle and separately at least one therapeutic agent; and applying to the cell an optical pulse sufficient to produce a nanobubble. Such methods may further comprise applying a dose of radiation to the cell.

The present disclosure provides, according to certain embodiments, compositions comprising at least one gold nanoparticle disposed adjacent to at least one therapeutic agent. Such compositions may further comprise a nanobubble disposed around the at least one gold nanoparticle.

The present disclosure provides, according to certain embodiments, systems comprising: a composition comprising at least one gold nanoparticle separate from and disposed adjacent to at least one therapeutic agent; and a laser disposed operable to the composition, the laser is capable of generating an optical pulse sufficient to create a nanobubble around the composition. Such systems, in some embodiments, may further comprise a detector for detecting the nanobubble. In other embodiments, such system may further comprise a radiation source.

While any cell capable of being targeted may be used, cancer cells are particularly suited for use according to the present disclosure. The term “cancer” refers to any of a number of diseases characterized by uncontrolled, abnormal proliferation of cells, the ability of affected cells to spread locally or through the bloodstream and lymphatic system to other parts of the body (e.g., metastasize), as well as any of a number of characteristic structural and/or molecular features. A “cancerous cell” or “cancer cell” is understood as a cell having specific structural properties, which can lack differentiation and be capable of invasion and metastasis. Examples of cancers are, breast, lung, brain, bone, liver, kidney, colon, and prostate cancer (see DeVita, V. et al. (eds.), 2005, Cancer Principles and Practice of Oncology, 6th. Ed., Lippincott Williams & Wilkins, Philadelphia, Pa., incorporated herein by reference).

The gold nanoparticles should have a size of about 1,000 nm or less, and be capable of converting electromagnetic radiation into thermal energy. In some embodiments, the gold nanoparticles have a size of about 10 nm to about 100 nm. The gold nanoparticles are excitable by an optical pulse resulting in creation of a nanobubble surrounding the nanoparticle. As used herein, the term nanobubble and refers to the transient vapor bubble that emerges around a nanoparticle when it is locally and transiently heated by exposure to electromagnetic radiation. The nanoparticle itself may not evaporate, instead acting as a heat source and heat accumulator in an intricate process of heat transfer and phase transition in the nanoparticle environment at nanoscale. The nanobubble expands rapidly to its maximal diameter and then collapses with its lifespan being longer than the duration of radiation pulse that feeds the energy to the bubble through the nanoparticle. The nanobubble's rapid expansion produces a localized mechanical and non-thermal impact that may result in damage or destruction to cellular components or to the cell itself. In addition, nanobubbles may be detected by one or more optical or acoustic detectors, allowing for the detection of nanoparticle location (e.g., at a particular cell).

The cell is also provided with a therapeutic agent. The terms “therapeutic agent” and “drug” and “agent” are used interchangeably herein to refer to a compound that, when present in a therapeutically effective amount, upon exposure to a site of action, produces a therapeutic effect, and whose site of action is located or whose effect will be exerted on the surface or inside target cells. By way of example, a therapeutic agent may be a chemical agent, such as an antibiotic or anti-cancer agent (e.g., doxorubicin, paclitaxel, etc.), a polypeptide, a protein, or a nucleic acid (e.g., DNA, RNA, siRNA, and the like). In some embodiments, a therapeutic agent can be a cytostatic or cytotoxic drug, a genetically-active material and a signal-activating material. In further embodiments, the cytostatic or cytotoxic drug can be free or encapsulated. Examples of the cytostatic or cytotoxic drug include, but are not limited to, cisplatin, doxorubicin, paclitaxel, and 5-furourocil. Furthermore, in some embodiments, clinically-approved drugs can be introduced (e.g., administered by a patient) under reduced doses.

In some embodiments the therapeutic agent may encapsulated to provide delivery of the therapeutic agent. For example, the agent may be provided in liposomes, micelles and other laminar constructs known in the art. The encapsulation should be capable of being disrupted by a nanobubble.

In order to target the gold nanoparticle and therapeutic agent to a particular cell or tissue, in some embodiments, the nanoparticle and therapeutic agent (or encapsulated therapeutic agent) may be functionalized with a targeting agent. Suitable targeting agents are capable of localizing a nanoparticle or therapeutic agent to a particular target cell. Examples of suitable targeting agents include, but are not limited to, antibodies, aptamers, and peptides.

In other embodiments, the at least one gold nanoparticle and the at least on therapeutic agent form a cluster in the cell. For example, after administering separately an encapsulated drug and gold NPs they will assemble into a cluster within the cell through biologically-supported co-localization of both components. The co-localization of GNPs and drug will be provided by cellular mechanisms such as the membrane receptor co-localization or/and receptor-mediated endocytosis.

In some embodiments, the GNPs may range in size from about 20 nm to about 60 nm, at which GNPs exhibited good internalization and clustering in cancer cells. These sterile GNPs are commercially available and showed little or no toxicity in vitro and in vivo during our preliminary studies. Larger GNPs (e.g., >100 nm) cannot be easily internalized by cells. GNPs smaller than 10 nm are rapidly cleared by organism and therefore cannot reach the tumor.

In certain embodiments, the encapsulated drugs among three clinically proven medications for HNSCC: paclitaxel, cisplatin, and doxorubicin can be used. For instance, in some embodiments, we can use our well-established small (15-25 nm) PEG-PE micelles as carriers of insoluble paclitaxel, and commercially available drugs encapsulated into about 90-120 nm liposomes as carriers of soluble doxorubicin (Doxil) and cisplatin (Lipoplatin).

In certain embodiments, two separate conjugates with tumor-specific antibodies may be introduced to the cell concurrently. An advantage of this method is an ability to personalize and independently tune the doses of GNPs and therapeutic agents in order to optimize the cluster formation and the therapeutic effect.

In some embodiments, several FDA-approved and clinically-validated for HNSCC antibodies, C225 (Erbitux), Panitumumab may be used as the targeting agent. To increase the targeting efficacy and specificity, in other embodiments, additional targeting agents, such as CD147, can be used. In one embodiment, an antibody may be covalently conjugated with GNPs. Such conjugates are very stable and biologically safe, with a shelf life of up to 1 year. Their targeting efficacy and low toxicity in vitro and in vivo have been shown from the examples discussed later.

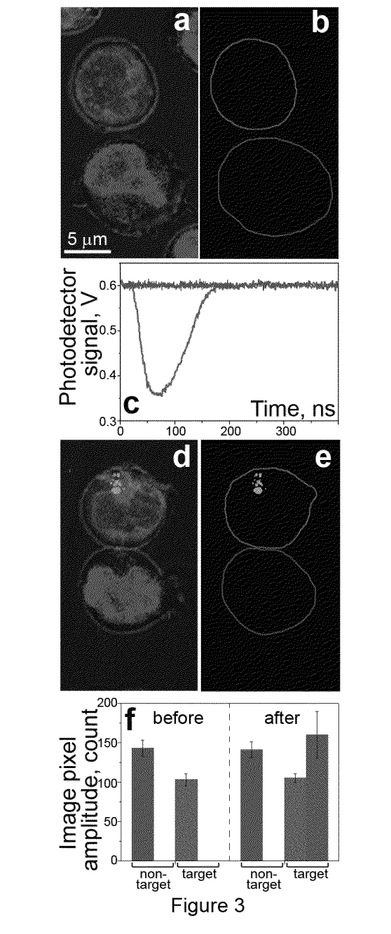

In certain embodiments, intracellular clusters of 10-50 tightly aggregated and mixed GNPs and drug nanocarriers in vivo occurs in several steps: (1) systemic i.v. injection of GNP-drug nanocarrier conjugates; (2) the leaky vasculature typical of tumors and the small size of GNPs (about 20-60 nm) will enable them to reach the close proximity to all tumor cells with the help of an this effect called “enhanced permeability and retention;” (3) antibodies conjugated with GNPs stimulates the maximal accumulation of GNPs at the membranes of the cells with the maximal level of the expression of target molecule (e.g., EGFR); (4) the receptor-mediated endocytosis of the membrane-accumulated GNPs and drug nanocarriers internalizes GNPs and nanocarriers and finally concentrates them into clusters in endo-lysosomal compartments (See e.g., FIGS. 3b-c and 7a).

As noted above, the gold nanoparticle can form a nanobubble when excited by electromagnetic radiation (e.g., an optical pulse). In general, suitable optical pulses are sufficient to create nanobubbles from nanoparticles that have been localized to a target cell. Suitable optical pulses may be provided by a laser pulse in the visible or near-infrared range of, for example, from about 100 fs to about 10 ns. In some embodiments, the optical pulses may be provided by a laser pulse in a range of from about 10 ps to about 100 ps.

In certain embodiments, the method further comprises applying a dose of radiation to the cell. Clinically-approved radiation sources can be used under reduced dose of the radiation in some embodiments.

To provide the maximal localized concentration of the released drug, the time interval between laser and radiation may be minimal. In some embodiments, the radiation is applied within about 48 hours after the optical pulse application. In a further embodiment, the radiation is applied within about 6 hours after the optical pulse application. In another further embodiment, the radiation is applied within about 3 hours after the optical pulse application. In a further embodiment, the radiation is applied within about 1 hour after the optical pulse application.

Intracellular amplification of chemotherapy and radiotherapy is achieved in these and other embodiments by administrating optically-absorbing nanoparticles and electromagnetic radiation together with drugs and radiation. Combined synergistic action of drug and nanobubbles results in a novel quadra-therapeutic mechanism. Thus, the invention, in some embodiments, is a combination of several processes and materials and improves the existing processes and products.

The quadratherapeutic mechanism of some embodiments of the present disclosure is localized to specific cells by separate targeting and administration of four components: standard encapsulated drug, radiation, GNPs, and short optical pulses. The quadratherapeutic mechanism of these embodiments comprises: (1) a mechanical effect wherein PNBs selectively impact disease-specific cells; (2) a chemotherapeutic effect that is selectively enhanced only in disease-specific cells by PNBs through the intracellular localized release of the encapsulated drug at high concentration; (3) a radiotherapeutic effect that is selectively enhanced only in disease-specific cells by clusters of plasmonic nanoparticles; and (4) the co-localization of the three above therapeutic mechanisms, which provides further amplification of the therapeutic effect with a single cell selectively due to the synergistic intracellular interaction of those mechanisms.

The multifunctional therapeutic effect of some embodiments is achieved in a procedure comprising the steps of (1) introducing into a cell at least one gold nanoparticle and separately at least one therapeutic agent; (2) applying to the cell an optical pulse sufficient to produce a nanobubble; and (3) administration of radiation.

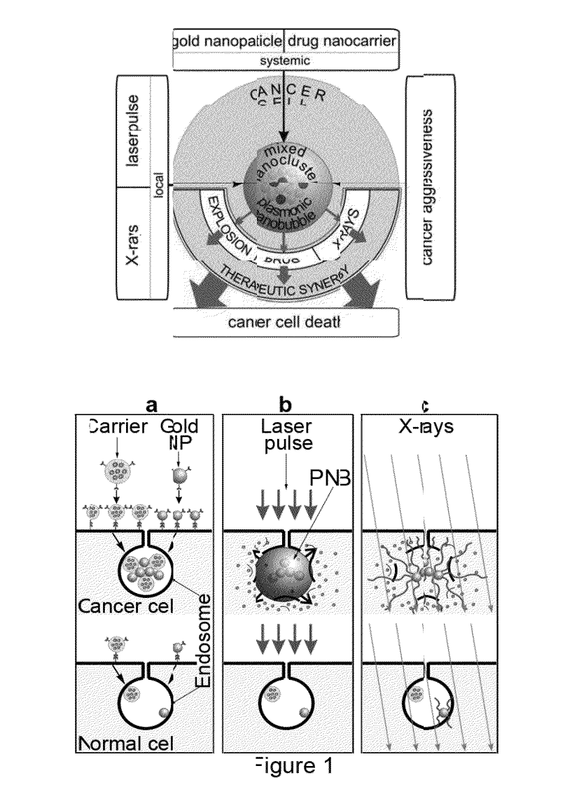

The first step of some embodiments comprises administration of drugs and GNPs (see FIG. 1a). GNPs and drug (encapsulated in carriers like liposomes, micelles or other carriers known in the art) are administered separately. In some embodiments, instead of or together with a drug, several agents can be administered—such as other drugs, genetic materials and/or diagnostic agents. To enhance their uptake by disease-specific cells, the nanoparticles and carriers of some embodiments may be functionalized with the disease-specific vectors (antibodies, aptamers, peptides or other disease-specific vectors known in the art and selected for a target disease). This provides formation of large clusters with nanoparticles being mixed with nanocarriers, preferably in disease-specific cells (see FIG. 1a) due to the mechanism, for example, of receptor-mediated endocytosis. The formation of the mixed cluster may be taken advantage of in the processes of some embodiments to provide further therapeutic effect in additional steps.

The process of some embodiments further comprises a second step, which comprises application of an optical pulse (see FIG. 1b). A short optical pulse—such as, for example, a laser pulse of specific duration (from about 100 fs to 10 ns), wavelength (visible and near-infrared) and fluence (above the generation threshold of PNBs in disease-specific cells)—is applied externally or through an optical guide in order to generate PNBs in the mixed clusters. Generation of PNBs in accordance with these embodiments may result in (1) immediate mechanical disruptive damage to the target cell and in (2) the release of the molecular cargo (drug or another cargo) from its carriers (like liposomes or else) into the cellular cytoplasm due to the disruption of nanocarriers by PNBs. PNBs of these embodiments disrupt not only the membranes of capsules but also eject the drug into the cytoplasm. This creates the high localized intracellular dose of the drug and thus will increase its therapeutic effect only in disease-specific cells because no PNBs will be generated in normal cells, and thus the drug will not be released in normal cells (FIG. 1b). In certain embodiments, the laser pulses may comprise near-infrared short laser pulses to be applied at low, physiologically safe doses (for example, doses comparable to, e.g., the current ANSI skin safety limits) in order to generate PNBs only in cancer cells (by using clusters of GNPs). The second step of these embodiments may therefore reduce the damage to normal cells and tissues. Furthermore, in some embodiments, the drug dose can be reduced compared to the current therapeutic dose, which may also reduce the damage to normal cells and tissues. In some embodiments, near-infrared laser pulse has the sufficient depth of tissue penetration up to 10 mm and can be delivered even deeper, up to 300 mm, with standard optical catheters and endoscopes.

The process of some embodiments further comprises a third step, which comprises administration of radiation (see FIG. 1c). In these embodiments, an external dose of radiation (gamma-rays or x-rays) may be administered within a specific time after the second step, administration of laser or other optical pulses. Clusters of GNPs may locally amplify the intensity of the radiation. This alone increases the effect of radiotherapy (see FIG. 1c). This may, in some embodiments, allow a reduction in the external dose of the radiation compared to the current therapeutic doses. In addition, in some embodiments, co-localization of the amplified radiation and the high local concentration of the released drug may provide significant benefits. Their interaction will, for example, further increase the efficacy of chemo- and radio-therapies. Such increase occurs in disease-specific cells, not in normal cells or in the whole body.

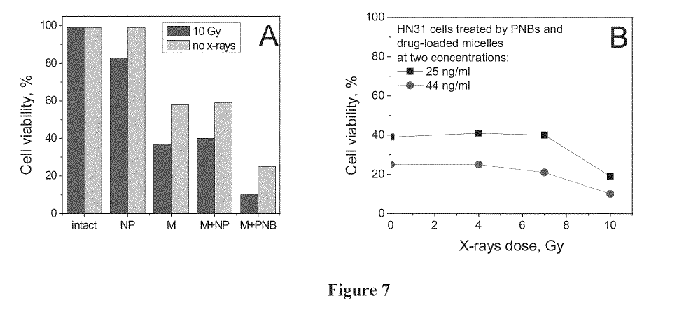

In some embodiments of the present disclosure, the combination of the above three steps may result in synergistic enhancement of mutual therapeutic effects of chemo-and radio-therapy in disease-specific cells. This makes it possible to realize synergy of drugs and radiation in disease-specific cells while sparing normal cells and organs. The methods of these embodiments provide for cell-level chemo- and radio-therapies with high selectivity and speed. See, e.g., FIG. 7.

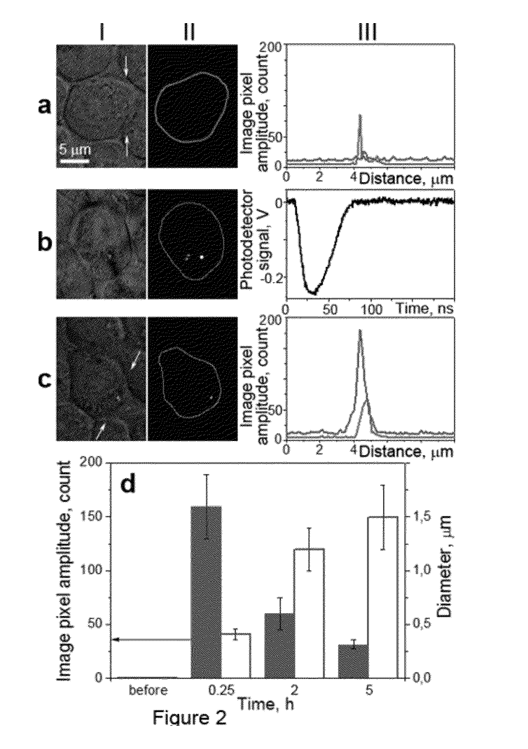

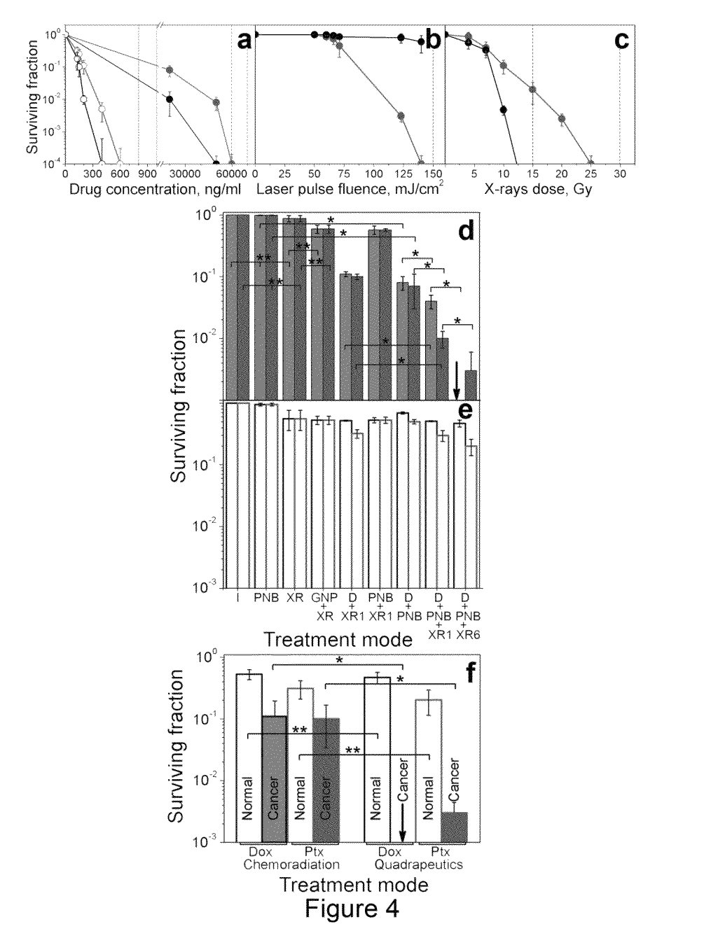

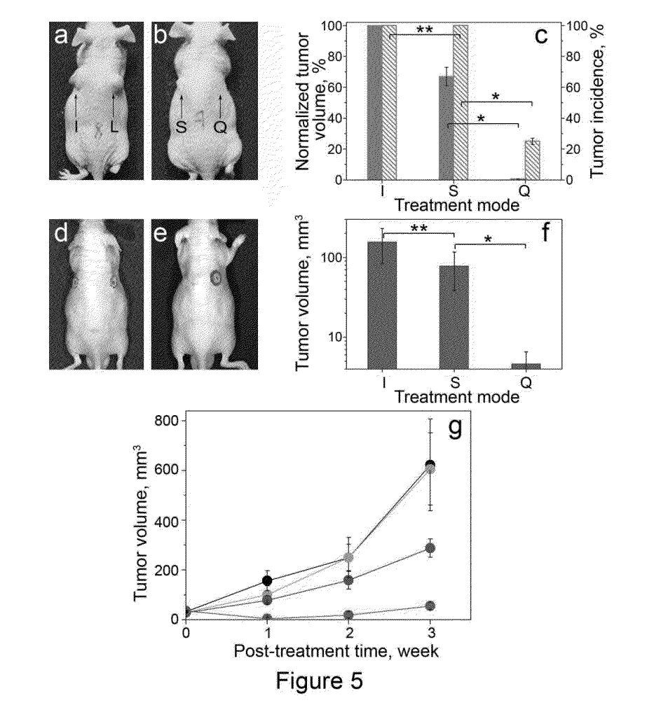

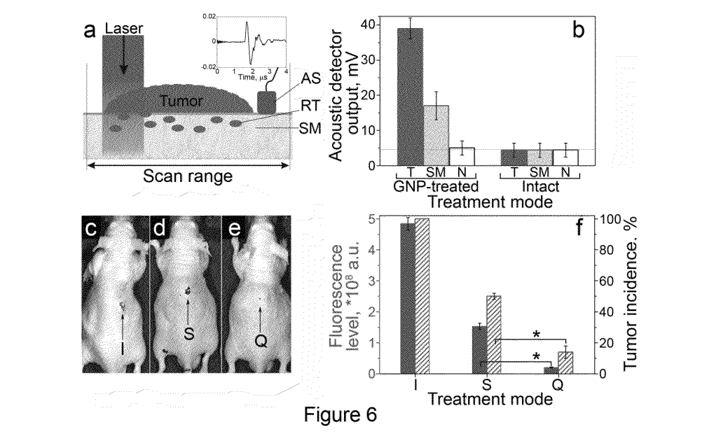

The coherence of the in vitro (see, e.g., FIG. 4) and in vivo (see, e.g., FIGS. 5 and 6) therapeutic data represents a solid proof of principle for the quadrapeutics. Its therapeutic action can be understood from the intracellular physical process, PNB (see, e.g., FIG. 2b): this explosive nano-event almost instantaneously creates the high localized intracellular concentration of the released drug whose interaction with locally-amplified by gold nano-clusters X-rays results in the radical acceleration and enhancement of the therapeutic effect of drugs and X-rays with a rapid and strong “knock-down” of a tumor within the first week after a single treatment (see, e.g., FIG. 5g). In contrast, traditional chemo- and chemoradiation treatments slowly build up their therapeutic effect with time and do not achieve the efficacy of quadrapeutics. In certain embodiments, the therapeutic effect of quadrapeutics can be further enhanced over time by applying the quadrapeutics a few times, e.g., with a 7-10 day intervals, similar to current chemo- and radiation therapeutic protocols. In some embodiments, the high therapeutic efficacy, speed and selectivity, and diagnostic sensitivity of quadrapeutics result from the following processes:

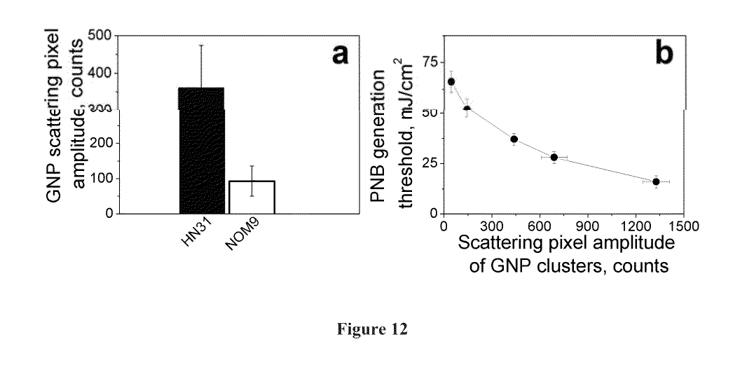

(i) Endocytosis creates cell-killing GNP-drug nanocarrier clusters following the separate administration of clinically-validated and safe colloidal GNPs and encapsulated drugs (which also eliminates the need for developing and approving new therapeutic complexes). Normal cells cannot build a sufficiently large cluster from fewer, non-specifically internalized GNPs and drug nanocarriers (see, e.g., FIGS. 1a, 12a, and 16). The PNB generation threshold laser fluence is relatively low for the large GNP clusters in cancer cells and high for small clusters in normal cells (see, e.g., FIGS. 1b and 12b). Therefore, the exposure of the cells to a low laser fluence results in PNBs only in cancer cells;

(ii) Intracellular PNB provides high intracellular concentration of the released drug (see, e.g., FIGS. 2c and 3). No current method of drug delivery can provide the combination of therapeutic efficacy, safety, specificity and short treatment time at cell level demonstrated by quadrapeutics (See, e.g., Table 1 in the Examples).

The slow diffusive release of the drug from non-specifically internalized nanocarriers in normal cells should not induce significant toxicity due to the radical (30-40 fold) reduction in drug doses (see, e.g., FIGS. 4e and 4f); and

(iii) Intracellular co-localization and synergy of the released drug and the amplified X-rays further enhances the therapeutic effect only in cancer cells (see, e.g., FIG. 4).

In certain embodiments, the method further comprises detecting acoustic responses.

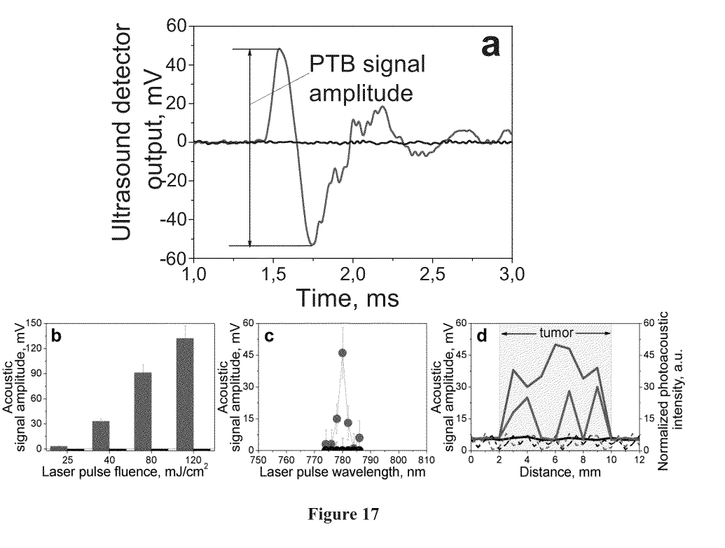

Acoustic emission by PNB allows for a rapid and highly sensitive diagnosis (see, e.g., FIG. 6b). Current intra-operative diagnostic methods require biopsy and are slow and often inaccurate. While the PNB method is technically close to photo-acoustics, the latter, unlike PNBs, has lower sensitivity (see, e.g., FIG. 17c), due to the weak acoustic emission of GNPs compared to that of PNBs. The acoustic detection of PNBs is also advantageous over optical intra-operative diagnostic methods, which are less sensitive and specific.

In some embodiments, these and other beneficial effects are achieved by synergistically amplifying two already existing therapeutics only in disease-specific cells, while sparing normal cells and organs.

In some embodiments, the methods described above can be used as an intra-operative diagnosis for the specific disease.

In other embodiments, the methods described above can be used as an adjuvant treatment of the specific disease.

Furthermore, the methods of some embodiments provide triple amplification of the therapeutic effect through, for example, any one or more of three interactions that may take place: (1) interaction of laser pulse with GNP clusters may induce transient PNBs whose mechanical, not thermal, effect will release the drug into cellular cytoplasm and will generate acoustic waves for tumor detection and guidance of the treatment; (2) interaction of the radiation with GNP clusters may induce local secondary electrons that will generate local radiation in cancer cell; and (3) interaction of radiation with the released drug may enhance cellular damage.

Compared to standard chemo- and chemoradiation therapies, quadrapeutics will allow significant reduction of therapeutic doses and treatment time without losing the therapeutic efficacy. This makes the treatment safer, simpler and more reliable compared to the current chemo- and chemoradiation therapies. The therapeutic efficacy and safety of current targeted therapeutic nanocarriers are limited by: (i) slow diffusive release mechanisms that “leak” the drug en route and in cancer cells without creating its high intracellular concentration fast enough, (ii) significant non-specific uptake by normal cells due to the high doses of nanocarriers (e.g., one-two orders of magnitude higher than those applied in quadrapeutics), and (iii) the failure to discriminate cancer cells from normal cells when using external energy modalities alone or in combination with GNP, or complex theranostic nanocarriers. Some nanocarriers are marketed as “nanobubbles”, but being the particles they have all the limitations of the aforementioned nanocarriers and none of the properties of PNBs. PNBs also differ from macro vapor (cavitation) bubble-based therapies, which are not cancer cell-specific and also require high drug doses.

GNPs alone enhance radiotherapy, but at the cost of high GNP doses, three to four orders of magnitude higher than in quadrapeutics. At lower GNP doses, the therapeutic gain is low (see, e.g., FIGS. 4d and 4e, modes GNP+XR). In certain embodiments, GNPs can also carry a drug and then amplify X-rays, but in the absence of intracellular co-localization of the drug and X-rays, their therapeutic effect requires 400-fold higher drug concentration (compared to our experiments) and has low cancer cell selectivity.

Laser microsurgery and thermal therapy employ bulk photothermal effects that require high optical doses and long exposure times, four to six orders of magnitude higher than those in quadrapeutics, and thus have low cancer cell specificity and often do not prevent tumor recurrence. GNPs improve the efficacy of photothermal therapy, but their high doses and non-specific uptake coupled with thermal diffusion do not improve cancer cell specificity or lower systemic toxicity. In contrast, our novel method of PNB generation with a short laser pulse allows combination of clinically-validated gold colloids (instead of less safe, engineered, near-infrared GNPs) with safe, deep-penetrating near-infrared light (e.g., up to 10 mm or up to 300 mm with standard optical catheters) and reduces the therapeutic optical dose by several orders of magnitude compared to other GNP-based therapies.

An example procedure using the above-discussed features of some embodiments follows:

1. Diagnosis-specific (target-specific) vectors are determined and conjugated to GNP and to the capsule containing the molecular cargo.

2. Conjugates of GNPs and capsules with drug are independently (separately) targeted (administered) in specific proportion.

3. As a result of the two former steps GNPs and capsules will be co-localized in their targets (cells, tissues), for example through endocytosis, and will form an aggregate (e.g., a cluster) containing both types of agents in endosomes of the target cells or at the membranes of target cells.

4. Non-specifically targeted agents will not form the above described multi-component clusters and non-coupled agents will be removed through various clearing processes.

5. The targeted zone(s) or volume(s) will be exposed to a short pulsed optical radiation (e.g., a laser) with parameters that provide generation of PNBs in multi-clusters of GNPs and capsules. PNBs will disrupt mechanically the capsules and thus will release or eject locally the molecular cargo that will interact with the target (for example, by release into cellular cytoplasm), thus providing localized high intracellular concentration of the released drug. This will provide a highly specific chemotherapeutic effect, and, in addition, PNBs will mechanically disrupt the components of the target cells (membrane, cytoskeleton etc.) thus providing additional therapeutic effect through mechanical ablation of the cells. It will also overcome drug resistance of, e.g., cancer cells.

6. After exposure to the optical radiation (e.g., laser pulses), the same area is exposed to a single dose of radiation. Clusters of GNPs will locally increase the intensity of the radiation and thus will locally amplify the dose of radiotherapy received by the cell with cluster(s) of metal nanoparticles. The time interval between laser and radiation treatment should be minimal in order to provide the maximal localized concentration of the released drug.

As a result, the three different therapeutic mechanisms in this example procedure will be selectively activated in individual target cells while the normal tissues and cells will not be exposed to the released drug and to the enhanced radiation, and the interaction of these mechanisms will synergistically enhance the overall therapeutic effect on target cells.

Another example comprises the application of paclitaxel-loaded 14 nm micelles, GNPs, PNBs and x-rays according to FIG. 7, with the results shown therein in FIGS. 7A and 7B.

In some embodiments, delivery of nanoparticles to a disease site may be improved, including by faster delivery and at higher concentrations, by the employment of any of various techniques, including, for example: extracorporeal treatment; topical application to surfaces associated with superficial pathologies; local injection into a zone or volume associated with deep tissue pathology.

In other embodiments, localized delivery of optical radiation (e.g., laser radiation) may be improved in order to overcome optical scattering and absorbance of tissues. For example, any of the following procedures (or combinations thereof) may be employed: extracorporeal treatment (for example, as with bone marrow or blood grafts); treatment of superficial pathologies where light can be directly delivered (for example, as in the case of lung, skin, and head and neck cancers; introduction of an optical catheter (for example, in treatment of pathologies associated with the walls of blood vessels); and fiber optic delivery (for example, to achieve localized treatment of deep tissues).

The present disclosure, in some embodiments, may comprise variations on the above-described approach. For example, the methods disclosed herein can be used in gene therapy. One example of a potential application of such embodiments to gene therapy is in treating cystic fibrosis. Patients with cystic fibrosis have a defective gene that encodes the code for a protein critical to a transfer of salts through the cell membrane in lungs. Conjugates of GNPs and liposomes with nondefective gene may be independently targeted to appropriate cells. Activation of GNPs will eject the genes from the liposomes in cells. The incorporation of the gene into the cells lining the lungs can stimulate the process of protein synthesis in large amounts.

In other embodiments, the methods of the present disclosure can be used for experimental delivery of drug or diagnostic agents in a controlled manner and at subcellular resolution. This method can be adapted to methods currently served by microinjection or ionophoesis. For example, the delivery of calcium release agonists into cells in vitro or in vivo can be used to map the effects of calcium release in discrete regions of the cell rather than whole cell treatments.

Furthermore, the methods of the present disclosure can, in some embodiments, be used for monitoring cell culture systems and animals to test the effect of encapsulated drugs and PNBs in vitro and in vivo, respectively.

To facilitate a better understanding of the present disclosure, the following examples of certain aspects of some embodiments are given. In no way should the following examples be read to limit, or define, the entire scope of the invention.

### EXAMPLES

Generation and Detection of PNBs

Generation of PNBs in vitro. A PNB is the localized transient evaporation of liquid around a laser pulse-heated GNP that results in a non-stationary expansion and collapse of a vapor nanobubble in nanoseconds. For PNB generation, we applied a single 70 ps laser pulse (PL-2250, Ekspla, Vilnius, Lithuania) at the wavelengths of 532 nm and 780 nm, using our novel method of non-stationary optical excitation of GNPs. This method used a single laser pulse and allowed the generation of PNBs around clinically-validated gold colloids in the near-infrared spectral region where tissue transparence is maximal and the depth of optical penetration is up to 10 mm. The diameter of the excitation laser beam was 220 μm in the in vitro study. Single PNBs were detected in vitro in individual cells through their optical scattering time-response (FIG. 2b-III).

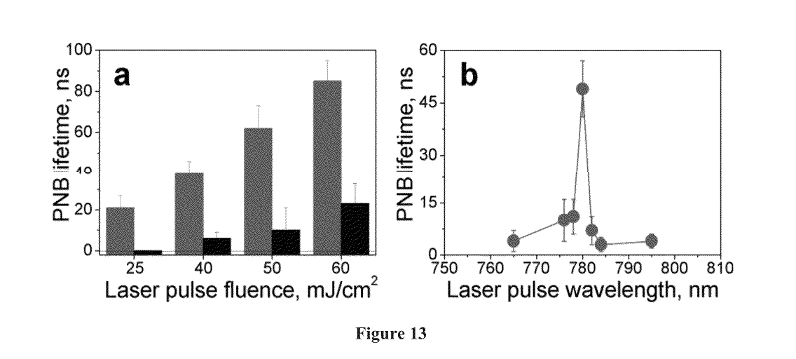

The cancer HN31 and normal NOM9 cells were incubated with GNP-C225 conjugates as described above. The GNP cluster-threshold mechanism provides the ultimate cancer cell specificity of PNBs that were generated at a low fluence, sufficient only to induce PNBs in cancer cells but not in normal cells in vitro (FIG. 13a). The PNB diameter and lifetime were easily controlled via laser pulse fluence (FIG. 13a) with very high, 2-4 nm wide, spectral selectivity (FIG. 13b). The PNB generation threshold fluence (laser energy per square centimeter) for HN31 cancer cells was approximately 27 mJ cm⁻², which is close to the ANSI safety limits and exceeds the PNB threshold for large GNP clusters, but was insufficient to generate PNBs around smaller clusters or single GNPs in normal cells.

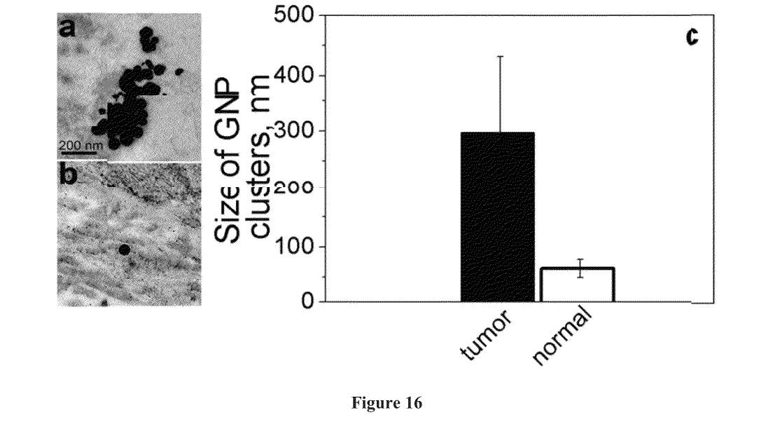

PNB generation in vivo. The GNP cluster-threshold mechanism of PNB generation provides the ultimate cancer cell specificity of PNBs (FIG. 17) via the formation of the largest GNP clusters only in cancer cells, through receptor-mediated endocytosis of GNPs (FIGS. 16a-c). The selectivity of PNBs generated in vivo was evaluated with a 70 ps laser pulse at different laser pulse fluences (FIG. 17b) 24 hours after the systemic injection of GNP-C225 conjugates in mice. The diameter of the excitation laser beam was 470 μm in this study. The maximal diameter of the PNB was measured in vivo through the acoustical responses (FIG. 5a). The PNB diameter was easily controlled via laser pulse fluence (FIG. 17b) with very high, 2-4 nm wide, spectral selectivity (FIG. 17c).

Single PNBs were detected in vitro in individual cells via their optical scattering as a time-resolved optical scattering image (see, e.g., FIG. 2b-II) and a time-response (FIG. 2b-III). Images were obtained with the pulsed probe beam (576 nm, 70 ps, 0.1 mJ cm⁻²). A PNB image showed the location of the PNB. The maximal diameter of the PNB was simultaneously measured with the optical scattering time-response in vitro (FIG. 2b-II) and acoustic response in vivo (FIGS. 6a and 17a). We focused an additional continuous laser beam of very low power (633 nm, 0.05 mW, 05-STP-901, CVI Meller Griot, Albuquerque, N. Mex.) on the cells. The PNB-induced scattering of the probe beam decreased its axial intensity, thus producing a time-response of a PNB-specific shape (FIG. 2b-III). The duration of this response at half of its maximum was measured as the PNB lifetime that correlates to the maximal diameter of the PNB. Real-time remote acoustic detection of PNBs in animals used a 2-mm ultrasound transducer XMS-310 (10 MHz, Olympus NDT Inc., Waltham, Mass.) with pre-amplifier (Ultrasonic Preamp 5676, Olympus NDT Inc., Waltham, Mass.). All hardware was assembled on an inverted optical microscope (Zeiss Axiovert 200) and was operated by a PC through custom software modules developed on a LabView (National Instruments Corporation, Austin, Tex.) platform.

Cells

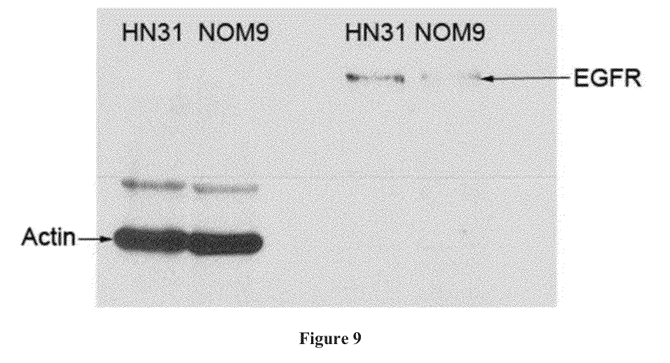

We used multi-drug resistant HN31 squamous carcinoma cells (associated with head and neck cancers) which are expressed by the epidermal growth factor receptor (EGFR) and immortalized normal human oral kerotinocyte NOM9 cells which have a 2.8 times lower level of EGFR expression than HN31 cells. (See FIG. 9). Both cell lines were obtained from the Johns Hopkins Genetic Resources Core Facility.

The HN31 head and neck squamous carcinoma cells were used in in vitro and in vivo studies. Normal epithelial human oral kerotinocyte NOM9 were used in in vitro therapeutic studies. The suspension model used other EGFR-negative cells, Jurkat (J32) suspension cell line, because adherent NOM9 cells had low survival after the trypsinization and multiple staining procedures employed in the suspension experiments. Pixel image amplitudes of dye-specific fluorescence and optical scattering were measured locally in individual cells for at least 150 cells per sample, by using image cytometry with a laser scanning confocal microscope LSM710 (Carl Zeiss MicroImaging GmbH, Germany). Population-averaged metrics were obtained to characterize the drug release (green fluorescence), cell type (red and blue fluorescence), integrity of the PNB-treated cells (Calcein Red fluorescence) and GNP uptake (scattering).

GNPs and Tumor Targeting

GNP clusters in cells in vitro. The GNP cluster formation was verified in vitro by using HN31 squamous carcinoma cells and normal human NOM9 cells. The cells were grown in Ibidi μ-Slide Angiogenesis (μ-Slide Angiogenesis, Ibidi LLC, Martinsried, Germany) and incubated for 24 hours at 37° C. with 60 nm gold nanoparticles (GNP) conjugated with the anti-EGFR antibody C225 (2.4×10¹⁰ GNPs ml⁻¹). The unbounded GNPs were washed off prior to cell imaging and laser treatment (70 ps, 532 nm, 0-75 mJ cm⁻²). Thus, only GNPs internalized through receptor-mediated endocytosis were detected and used in cells as a source of PNBs under laser pulse exposure.

For detection of GNP clusters in living HN31 and NOM9 cells, the LSM 710 laser confocal microscope was used in scattering mode as described above. The pixel image amplitudes (FIG. 2a) were measured locally in each individual cell for at least 150-180 cells per sample (3 samples were used for each type of cell), and were then analyzed as the population-averaged metrics of GNP clusters (FIG. 12a): while the cancer (HN31) cells show significant clustering of GNPs, single GNPs or only small clusters were observed in the normal (NOM9) cells.

Next we measured the PNB generation threshold fluence in HN31 cells as a function of the GNP cluster size measured through its scattering image amplitude (FIG. 12b): it was the lowest for the largest clusters and increased several fold for smaller clusters and single GNPs.

We used 60 nm solid gold spheres (colloidal gold, the type of GNPs used in clinic for more than 50 years) conjugated with the antibodies against EGFR, Erbitux (ImClone Systems Inc., Branchburg, N.J.) (C225). The use of gold colloid in near-infrared as efficient PNB sources eliminated the need for specifically engineered near-infrared nanoparticles such as nanoshells or nanorods. The GNP conjugates were prepared by BioAssayWorks LLC (Ijamsville, Md.) and were administered at the concentration of 2.4×10¹⁰ NPs mL⁻¹ in vitro and 0.8 μg g⁻¹ of body weight in vivo (via intravenous injection) simultaneously with drug-loaded nanocarriers. Receptor-mediated endocytosis of both conjugates resulted in the formation of mixed endosomal clusters of GNPs and nanocarriers. After incubation with cells in vitro, free GNPs and nanocarriers were washed off cells prior to the laser treatment. GNPs were imaged and quantified with optical scattering confocal microscopy in vitro (FIGS. 2 and 12a).

GNP targeting in vivo. The evaluation of GNP clustering in vivo was done using TEM microscopy (Hitachi H-7500 Electron Microscope) (FIGS. 16a-c). Twenty-four hours after the systemic injection of GNP-C225 conjugates (0.8 μg g⁻¹), the tumor and adjusted normal tissues were extracted and prepared using the standard technique for TEM imaging. The big GNP clusters were observed only in the tumor and just small clusters or single GNP were detected in normal tissue. The non-specific uptake of GNP-C225 conjugates by normal cells resulted in single GNPs (FIGS. 12a and 16) or much smaller clusters that showed a much higher PNB generation threshold fluences compared to the lower PNB threshold for large GNP clusters in HN31 cells (FIGS. 13a and 17b).

Synthesis and Characterization of Drug Nanocarriers

Micelle preparation. Paclitaxel was incorporated in mPEG₂₀₀₀-PE micelles by the lipid film hydration method. Briefly, 0.1 mg of paclitaxel (10 mg/mL in methanol) was mixed with a mPEG₂₀₀₀-PE solution in chloroform. The organic solvents were removed by rotary evaporation followed by freeze-drying. The film was hydrated with 10 mM phosphate-buffered saline (PBS), pH 7.4 at room temperature and vortexed for 5 minutes to give a final lipid concentration of 5 mM. The unincorporated drug was removed by filtration of the micelle suspension through 0.2 μm membrane filters.

Calcein Green-loaded liposomes. Calcein Green-loaded liposomes were employed to study the release process.

Liposomes were first prepared by the lipid film hydration method. A chloroform solution of ePC and cholesterol (70:30 molar ratio) was evaporated by rotary evaporation followed by freeze-drying. The film was then hydrated in 1 mL 50 mM Calcein Green solution (Calcein Green AM (C34852, Invitrogen, Carlsbad, Calif.)). The resulting multilamellar liposome solution was then extruded 11 times through a 200 nm pore sized Nuclepore polycarbonate membrane (Whatman) using an Avanti hand extrusion device (Avanti Polar Lipids). After extrusion, the extraliposomal calcein buffer was removed by gel filtration on a BioGel 1.5M. The size of the Calcein-loaded liposomes was 149.23±23 nm respectively. The conjugation of the liposomes with antibody 2C5 to cancer-specific nucleosomes, did not change the liposome size significantly.

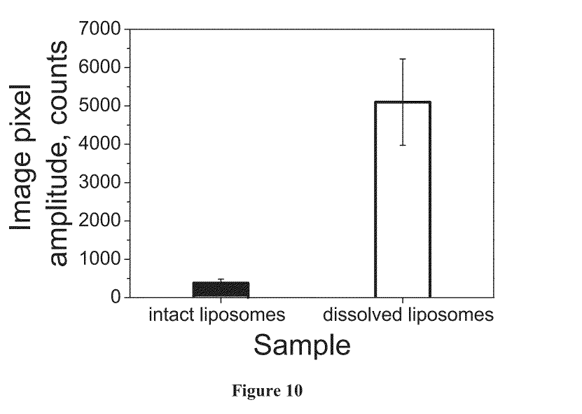

The fluorescence signals of both intact Calcein Green-loaded liposomes and those dissolved with alcohol, were tested by using a LSM710 laser confocal microscope (Carl Zeiss MicroImaging GmbH, Germany). The liposome suspension was mixed with alcohol (10:1 ratio) and the thin (3 μm) samples of intact and dissolved liposomes were prepared between two pieces of glass. The high concentration of the dye in the liposomes caused significant quenching that dimmed its fluorescence in the intact liposomes (FIG. 10). In a suspension test, the liposomes that had been dissolved with alcohol caused an increase in the level of green fluorescence by 16 fold (FIG. 10). Three samples were prepared and imaged for intact and test groups.

Liposomes were functionalized with 2C5 antibody raised against cancer-specific nucleosomes and were incubated with cells simultaneously with GNP-C225 conjugates for 24 hours (37° C.). The optical absorbance of the Calcein Green dye at 532 nm was more than six orders of magnitude lower than that for GNPs and given the low fluence of the laser pulse, the dye did not influence the PNB generation. The high concentration of the dye in intact liposomes quenched the fluorescence below a detectable level. Free liposomes and GNPs were washed off cells prior to exposure of the cells to laser pulses.

Doxorubicin-loaded liposomes. Standard Doxil liposomes with doxorubicin, a water soluble drug, (Ben Venue Laboratories, Inc, Bedford, Ohio) were functionalized with the C225 antibody. Cells were incubated with liposomes simultaneously with GNP-C225 for 24 hours. Free liposomes and GNPs were washed off cells prior to exposure of the cells to laser pulses.

Paclitaxel-loaded micelles. The paclitaxel-loaded micelles were prepared by the method described above. The micelle diameter was 14.5±0.11 nm. They were functionalized with C225 (against EGFR) or 2C5 (against nucleosomes) antibodies and incubated with cells and GNP-C225 for 4 hours (37° C.). Free micelles and GNPs were washed off cells prior to exposure of the cells to laser pulses.

Synthesis of pNP-PEG₃₄₀₀-PE conjugate. In order to prepare antibody (mAb 2C5/mAb C225)-modified micelles/liposomes, we first conjugated the antibody to the distal tips of PEG blocks via p-nitrophenylcarbonyl (pNP) groups (using a pNP-PEG₃₄₀₀-PE conjugate) to form antibody-PEG₃₄₀₀-PE conjugate. Modification of drug-loaded mPEG₂₀₀₀-PE micelles or Calcein-loaded liposomes or Doxil with this conjugate was done using the post-insertion method. The pNP-PEG₃₄₀₀-PE was synthesized and purified according to a previously established method. Briefly, the DOPE was mixed with a 5-fold molar excess of PEG-(pNP)₂ in chloroform in the presence of triethylamine. Organic solvents were removed, the resultant pNP-PEG₃₄₀₀-PE micelles were separated from free PEG and pNP on a sepharose CL-4B column. The product pNP-PEG₃₄₀₀-PE obtained was freeze-dried and stored in chloroform at −80° C.

Preparation of antibody-PEG₃₄₀₀-PE conjugate and preparation of targeted-micelles and liposomes. The chloroform solution of reactive component, pNP-PEG₃₄₀₀-PE (32 molar excess over antibody) was evaporated and freeze-dried to form a film in a small test tube. The dried film was hydrated with 5 mM citrate buffered saline pH 5.5 containing 10 mg mL⁻¹ octyl glucoside followed by the addition of antibody solution in PBS pH 7.4 or water. The pH was adjusted to 8.0-8.5 with 100 mM phosphate buffer pH 8.5. The reaction was continued overnight at 4° C. The next day, the micelles were dialyzed against 1 L of 10 mM PBS, pH 7.4 using cellulose ester membranes with a cut-off size of 300 kDa. The amount of antibody in the antibody-PEG₃₄₀₀-PE conjugate was estimated by a bicinchoninicacid (BCA) protein assay with pure antibody as the standard. The drug loaded PEG₂₀₀₀-PE micelles (0.5 mL) were incubated overnight with antibody-PEG₃₄₀₀-PE conjugate (equivalent to 0.487 mg of antibody) to prepare targeted micelles. To prepare 2C5 or C225-targeted liposomes, 1 mL of liposomes were incubated overnight with antibody-PEG₃₄₀₀-PE conjugate (equivalent to 0.150 mg of antibody).

Characterization of micelles and liposomes. The micelle and liposome size (hydrodynamic diameter) was measured by dynamic light scattering (DLS) using a N4 Plus Submicron Particle System (Coulter Corporation, Miami, Fla., USA). The micelle/liposome suspensions were diluted with deionized, distilled water until a concentration providing a light scattering intensity of 5×10⁴ to 1×10⁶ counts/sec was achieved. The particle size distribution of all samples was measured in triplicate. The size of the Paclitaxel-loaded micelles was 14.5±0.11 nm. Antibody modification did not change the micelle/liposome size significantly.

The amount of Paclitaxel in the micelles was measured by reversed phase-HPLC. The micelles were diluted with the mobile phase prior to application to the HPLC column. The samples were analyzed by reversed phase-HPLC. A D-7000 HPLC system equipped with a diode array and fluorescence detector (Hitachi, Japan) and Symmetry C18 column, 4.6 mm×250 mm (Waters, Milford, Mass., USA) was used. The column was eluted with water/acetonitrile (30:70% v/v) at 1.0 ml min⁻. Paclitaxel was detected at 227 nm. The injection volume was 50 μL. All samples were analyzed in triplicate. The amount of Paclitaxel loaded in plain mPEG₂₀₀₀-PE and antibody-modified mPEG₂₀₀₀-PE micelles was found to be 0.1 mg mL⁻¹ and 0.08 mg mL⁻¹ respectively. The amount of Doxorubicin in liposomes was determined after the treatment of the liposome sample with 1% Triton-100 using plate reader (Synergy HT multimode microplate reader, BioTek Instrument, Winooski, Vt.) with 485/590 nm excitation/emission wavelengths.

Confocal imaging of cancer cells. The cancer HN31 cells were incubated with GNP-C225 conjugates (2.4×10¹⁰ GNPs mL⁻¹) and Calcein Green-loaded liposomes conjugated with 2C5 antibody during 24 hours at 37° C. The unbound GNPs and liposomes were washed off prior to laser treatment (70 ps, 532 nm, 40 mJ cm⁻²). Thus the cells were exposed only to the internalized GNP and liposomes during the follow-up generation of PNBs. A LSM 710 laser confocal microscope was used in fluorescence and scattering (under excitation with a 633 nm continuous laser) modes for detection and analysis of GNPs and liposome-specific green fluorescence in individual living cells before and from 10 minutes to 5 hours after the exposure to a laser pulse (FIG. 3a). The pixel image amplitudes were measured locally in each individual cell for at least 150-180 cells per sample (3 samples were studied) and were then analyzed as the population-averaged metrics of GNP cluster formation (FIG. 12a) and dye release (green fluorescence) (FIG. 3e).

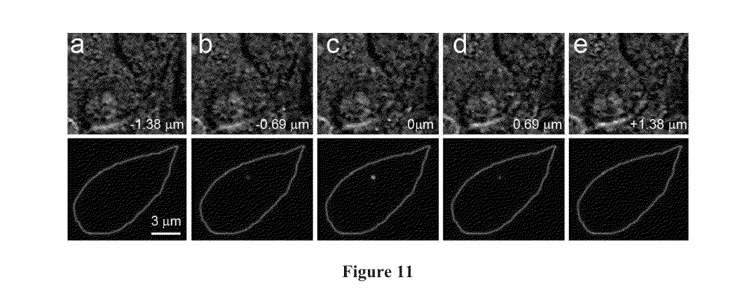

Three-dimensional confocal imaging of living cells was performed to evaluate the location and co-localization of liposome-associated dye release and GNP clusters in cells immediately after the exposure to the laser pulse. To do this, the LSM 710 laser confocal microscope was used in a Z-stack mode with 0.69 μm layer thickness (FIG. 11). The obtained images confirm the intracellular dye release via the mechanical disruption of the dye-loaded liposomes and cellular endosomes during PNB expansion and the ejection of the dye into the cytoplasm of cells where PNBs were generated.

Radiation Treatment

Living cells and animals were irradiated with X-rays with a RS 2000 machine (Rad Source Technologies, Inc., Suwanee, Ga.). In the case of combination treatment, the X-rays were administered from one to six hours after the laser treatment. A standard irradiation mode was used (160 kV, 25 mA, with a copper filter). The biological effect of these X-rays in cells and small animals was identical to that induced in humans with the higher energy of X-rays (in MeV range). The energy of X-rays determines the tissue penetration depth and does not generally influence the therapeutic mechanism. (See, e.g., McMahon, S. J., Mendenhall, M. H., Jain, S. & Currell, F. Radiotherapy in the presence of contrast agents: a general, figure of merit and its application to gold nanoparticles. Phys. Med. Biol. 53, 5635-5651 (2008); and Rose, J. H., et al. First radiotherapy of human metastatic brain, tumors delivered by a computerized tomography scanner (CTRx). Int. J. Radiat. Oncol. Biol. Phys. 45, 1127-1132 (1999)). All cells and animals received a single treatment.

Animal Models

Healthy, male athymic nude mice, age 8 to 12 weeks, were purchased from the National Cancer Institute-Frederick Cancer Research and Development Center (Frederick, Md.) and used in accordance with Animal Care Use Guidelines under the protocols approved by IACUCs of the UT MD Anderson Cancer Center and of Rice University. Two different established models of HNSCC were used.

Recurrent disease. This model used a reduced number of HNSCC cells: 180 000 of the in vitro pre-treated HN31 cells were injected on the mice flanks for the modeling of local recurrent disease.

Four treatment groups were analyzed: intact HNSCC cells (5 animals), HNSCC cells pre-treated with PNBs (without drugs or X-rays) (5 animals), HNSCC cells pre-treated with standard chemoradiation therapy (Doxil, 2 μg mL⁻¹ and X-rays, 4 Gy) (6 animals) and HNSCC cells pre-treated with quadrapeutics (Doxil, 2 μg mL⁻¹, GNP, 2.4×10¹⁰ particles mL⁻¹, laser pulse, 45 mJ cm⁻² at 24 hours after GNP administration, X-rays, 4 Gy, 6 hours after laser treatment) (4 animals). The tumors were characterized by their volume and the incidence rate at the stage (15 days) when the untreated tumors typically reached for a moribund stage. All animals were monitored on a daily basis. Tumor volume was estimated as half of the small diameter squared multiplied by the large diameter.

Bioluminescent imaging was performed with a highly sensitive, cooled CCD camera mounted in a light-tight specimen box, using protocols similar to those described previously. (See, e.g., Jenkins, D. E., et al. Bioluminescent imaging (BLI) to improve and refine traditional murine models of tumor growth and metastasis. Clin. Exp. Metastasis 20, 733-744 (2003); and Rehemtulla, A., et al. Rapid and quantitative assessment of cancer treatment response using in vivo bioluminescence imaging. Neoplasia 2, 491-495 (2000)).

Imaging and quantification of signals were controlled by the acquisition and analysis software Living Image. For in vivo imaging, animals were given the substrate D-luciferin by intraperitoneal injection at 150 mg kg⁻¹ in DPBS Dulbecco's Phosphate Buffered Saline (Invitrogen, Carlsbad, Calif., USA), and anesthetized (1-3% isoflurane). Mice were then placed onto the warmed stage inside the light-tight camera box with continuous exposure to 1-2% isoflurane. Imaging time was 10 s. Generally, two to three mice were imaged at a time.

Primary xenograft HNSCC tumors were induced s.c. by injecting 0.5 min of Luciferase-encoded HN31 cells and was grown to 3-5 mm. Tumors were quantified weekly via their volume (measured with a caliper) and Luciferase-induced bioluminescence (measured via small animal imaging system IVIS Lumina). One group received no treatment (6 animals), other three groups received the following single primary treatments: Quadrapeutic group (11 animals) received GNP-C225s (0.8 μg g⁻¹) and Doxil-C225 (1 mg kg⁻¹) via intra-venous concomitant injection. In 24 hours tumor areas (15×15 mm) were scanned with broad near-infrared laser pulses (780 nm, 45 mJ cm⁻²) and then after 6 hours were exposed to X-rays (4 Gy). PNB group (4 animals) received identical doses of GNP and laser pulses. Generation of PNBs in tumors was monitored with ultrasound detector during the laser scan (FIG. 6a). Chemoradiation group (11 animals) received identical doses of drug and X-rays as the quadrapeutic group. All animal were monitored for three weeks, the period that stably showed a moribund condition among untreated animals.

Tumor width and length were measured with digital calipers, and the tumor volume was calculated in mm³ as: Volume=(width)²×length/2 (See Rofstad, E. K. & Brustad, T. Tumour growth delay following single dose irradiation of human melanoma xenografts. Correlations with tumour growth parameters, vascular structure and cellular radiosensitivity. Br. J. Cancer. 51, 201-210 (1985)). Therapeutic efficacy was calculated as a ratio V1/V2, where V1—averaged tumor volume for animals in untreated group, V2—averaged tumor volume for animals in treated group. Animals were monitored for three weeks after the treatment and were euthanized on reaching a moribund condition.

MRD model. The tumors were xenografted with the HN31 cells as previously described. (See, e.g., Jiffar, T., et al. KiSS1 mediates platinum sensitivity and metastasis suppression in head and neck squamous cell carcinoma. Oncogene. 30, 3163-3173 (2011)). The tumors were induced on the mice flanks: the nude mice were anesthetized and 1×10⁶ HN31 encoded with GFP cells was injected using a 1-mL tuberculin syringe with a 30-gauge hypodermic needle. 14 to 17 days after the cell injection, when the tumors were already established (5-7 mm in diameter), the GNP-C225 (0.8 μg g⁻¹ of body weight) and/or Doxil-C225 (1 mg kg⁻¹ of body weight) conjugates were injected into the anesthetized mice via the tail vein using an intravenous catheter and a 1-mL-insulin-syringe. Twenty-four hours after GNP and drug injection, the tumors were fully resected and the surgical margins were exposed to a scanning laser beam (70 ps, 780 nm, 45 mJ cm⁻², 470 μm diameter) to generate PNBs and to detect them via acoustic responses. Acoustic detection employed the generation of the pressure transients during the PNB expansion and collapse, complemented optical scattering detection, and, most importantly for the diagnostic application, provided the in vivo detection of PNBs in opaque tissue. The amplitude of the acoustic response was used as the PNB metric and was correlated to the optically measured lifetime of the PNB. The surgical wounds were then closed.

The local recurrence of HNSCC was monitored in the animals by visual observation on a daily basis. Also the small animal imaging of GFP fluorescence was performed with an IVIS Lumina (HN31 cells expressed Green Fluorescent Protein) Imaging System. The probability of tumor recurrence and GFP-fluorescence signals were analyzed. All animals were monitored for tumor growth on a daily basis. The incidence of tumor recurrence and the intensity of GFP-fluorescence signals were analyzed at a specific time interval after the surgery, 28 days, for three treatment modes: surgery (5 animals), surgery with adjuvant chemoradiation therapy (Doxil+X-rays) (6 animals), and surgery with adjuvant quadrapeutics (7 animals).

Therapeutic Efficacy and Statistics

The therapeutic effect was measured in vitro as short-term viability and long-term clonogenicity. In vivo studies used 4 to 11 animals in each group randomly assigned to groups for experiments. In vivo therapeutic efficacy was analyzed by three standard metrics: the tumor incidence rate, tumor volume and the intensity of the fluorescence (or luminescence) of encoded tumor cells. Two-tailed t-tests were used to compare groups for HNSCC cell fluorescence, tumor incidence and volumes. Statistical analyses were performed with Origin software (OriginPro8, OriginLab Corporation, Northampton, Mass.). P values<0.05 were considered statistically significant.

The PNB Mechanism of On-Demand Intracellular Release of an Encapsulated Payload

This was studied in the HNSCC HN31 cell line. Cancer cells were targeted by using the clinically-validated molecular target, epidermal growth factor receptor (EGFR) with a matching C225 antibody conjugated to 60 nm solid gold spheres (GNPs, also known as gold colloids). A high level of EGFR expression (FIG. 9) resulted in the endocytotic formation of large GNP clusters in cancer cells (FIG. 2a). Liposomal green fluorescent dye conjugated with 2C5 antibody co-targeted cancer cells. Confocal scattering and fluorescent imaging of HN31 cells after incubation with separately administered GNPs and liposomes revealed intracellular GNP clusters, but showed no fluorescence due to its quenching in intact liposomes (FIGS. 2a and 10).

Next, the cells were exposed to a single laser pulse of the relatively low fluence of 40 mJ cm⁻² at 532 nm to generate PNBs. PNBs were detected in individual cells with time-resolved optical scattering imaging that showed good intracellular co-localization with GNP clusters (FIGS. 2b-I and II), and an optical scattering time-response that showed PNB expansion and collapse within 50-60 ns (FIG. 2b-III). The duration of the response was measured as the main metric of a PNB, the lifetime. Immediately after the PNB's generation, re-imaged cells yielded a bright liposome-specific green fluorescence in sub-micrometer zones co-localized with the GNP clusters and PNBs (FIG. 2c). No released dye was observed in PNB-negative cells. Three-dimensional confocal images of the cells confirmed the dye release into cellular cytoplasm (FIG. 11). This indicated an almost instantaneous localized release of the dye due to the mechanical (not thermal) impact of the PNB that disrupted the internalized liposomes and endosomes and ejected the dye during the explosive PNB expansion, e.g., within approximately 30 ns (FIG. 2b-III). Small size of the PNB (200-400 nm for a PNB with a 60 ns lifetime) coupled with the high speed of the release resulted in a high intracellular concentration of the payload released in a very small volume. This release mechanism was quantified via image cytometry of the cells through the population-averaged diameter and pixel image amplitude of the local intracellular green fluorescence, which demonstrated its localized and rapid nature (FIG. 2d).

Cell Suspension Model for Evaluation of the Mechanism of PNB-Induced Release

The cancer cell specificity of PNB-enhanced release was evaluated in a mixed suspension of EGFR-positive HN31 cells and EGFR-negative J32 cells. J32 cells were obtained from ATCC. Both types of cells were incubated (24 hours, 37° C.) separately with GNP-C225 conjugates and Calcein Green-loaded liposomes conjugated with 2C5. After incubation, the unbound GNPs and liposomes were washed off. For cell identification, we used red fluorescent dye (CellTrace Calcein Red AM, C34851, Invitrogen Corporation, Carlsbad, Calif.) for the labeling of HN31 cells, and blue (DAPI, D1306, Molecular Probe Inc, Eugene, Oreg.) fluorescent dye for the labeling of J32 cells. After labeling, HN31 cells were trypsinezed and mixed with the labeled J32 cells prior to PNB generation with a short laser pulse of 70 ps, 532 nm, 40 mJ cm⁻². The mixed cell populations were imaged and analyzed before and after their exposure to the laser pulse by using the LSM 710 laser confocal microscope in tricolor configuration with red (Calcein Red AM), blue (DAPI) and green (Calcein Green AM) dyes (FIG. 3).

Cells were considered as red, blue or green-positive when their image pixel amplitude exceeded that of intact cells. Pixel image amplitudes were measured in each individual cell for at least 150-180 cells per sample (3 samples were analyzed) and were then analyzed as the population-averaged metrics of drug release (green fluorescence), cell type (red and blue), and integrity of the cells with laser-induced PNBs (red) (FIG. 3).

After washing off unbounded GNPs and liposomes, we first obtained a set of tri-color (red-blue-green) fluorescent images (FIGS. 3a and 3b) and then scanned the mixed cell suspension with broad pulsed laser beam (70 ps, 532 nm, 40 mJ cm⁻²) that simultaneously irradiated hundreds of red and blue cells.

The generation of PNBs in individual cells was monitored through optical scattering time-responses (FIG. 3c). We observed PNBs with a lifetime of 55-60 ns in HN31 (red) but none in J32 (blue) cells. The threshold nature of PNB prevented their generation in EGFR-negative cells because these cells did not form sufficiently large GNP clusters (FIG. 12a) and therefore had a higher PNB generation threshold fluence (FIG. 12b), above the level of the applied laser fluence.

The second tri-color imaging, obtained immediately after laser treatment, revealed the localized bright green fluorescence in HN31 (red) cells but none in J32 (blue) cells (FIGS. 3d and 3e). Image cytometry of 150-180 HN31 and J32 cells each before and after the laser pulse (FIG. 3f) showed (i) a similarity of the green fluorescent patterns to that in FIG. 2b, (ii) a high contrast of green fluorescence after PNB and (iii) high cancer cell specificity of the intracellular release of the liposomal payload. In all cells, the onset of the local green fluorescence correlated with PNB generation.

This unique combination of the nanosecond, on-demand, PNB-enhanced release, the high intracellular localization of the released payload and high cancer cell specificity can support efficient intracellular delivery of drugs and other molecular cargo.

Efficacy and Selectivity of the PNB-Enhanced Release of Soluble and Non-Soluble Drugs from Nanocarriers.

Short term response in vitro to co-localized administration of drug nanocarriers, GNPs and laser pulses. We used EGFR-positive HN31 (cancer) cells and EGFR-negative NOM9 (normal) cells. Drug carriers and GNP-C225 conjugates (2.4×10¹⁰ GNPs mL⁻¹) were separately administered to cells (for 24 hours with Doxil-C225 (Dox, 5 μg mL⁻¹) and for 4 hours with micellar Paclitaxel-C225 (Ptx, 0.065 μg mL⁻¹)) and were then washed off prior to laser treatment (70 ps, 780 nm, of 45 mJ cm⁻²). After incubation, GNPs and drug carriers were washed off. Thus the cells were exposed only to the internalized drug during the follow-up generation of PNBs. PNB lifetime was obtained for individual cells. The short-term viability was evaluated 72 hours after the treatment of samples as a complex viability parameter RRV that included the viability level V1 (measured in % with Trypan Blue exclusion test) and the cell concentration C: RRV=C/C₀*V₁,*100% , where C₀ is the cell concentration in the intact sample.

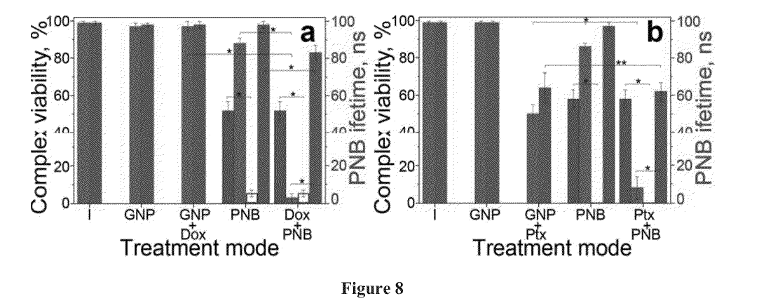

The ability of the PNB mechanism to overcome drug resistance and to reduce non-specific toxicity was evaluated with two common drugs and carrier types. Water-soluble doxorubicin was encapsulated into PEG-coated 90 nm Doxil liposomes that were conjugated with anti-EGFR antibody, C225 (L-Dox-C225). Non-soluble paclitaxel was loaded into 14 nm micelles conjugated to the same antibody (M-Ptx-C225). Each type of drug-loaded nanocarrier was administered separately with gold NP-C225 conjugates. Cancer (HN31) and normal (NOM9) cells were incubated identically with NP-C225 and nanocarriers. Drug doses were reduced to the level that provided relatively high viability of both cell types when treated with nanocarriers alone (without PNBs). Specifically, encapsulated doxorubicin was applied at the reduced dose of 5 μg mL⁻¹, resulting in a viability level cancer and normal cells close to those for intact controls (FIG. 8a). Encapsulated paclitaxel was applied at a reduced dose of 0.065 μg mL⁻¹, resulting in 50±5% % viability among cancer cells and 64±8% viability among normal cells (FIG. 8b).

After incubation, NPs and nanocarriers were washed off, the cells were exposed to a single laser pulse (70 ps, 532 nm, 40 mJ cm⁻²) and the PNB lifetime was obtained for individual cells. PNBs with 55-60 ns lifetimes were observed only in cancer cells (the lifetimes are shown in blue in FIGS. 8a and 8b). The cell concentration and viability were measured at 72 hours after the PNB generation as the complex viability parameter that was normalized by that in intact cells (FIG. 8). PNBs alone (without nanocarriers) did not significantly reduce the complex viability of cancer cells (FIGS. 8a and 8b). In contrast, cancer cells treated with drug-loaded nanocarriers and PNBs yielded a nearly complete loss of their viability. It dropped from 88±3% (PNBs alone) to 3±2% in the L-Dox-C225 treated cancer cells (FIG. 8a) and from 86±2% (PNBs alone) to 8±6% in M-Ptx-C225 treated cancer cells (FIG. 8b). PNB lifetime correlated well to the cell viability and therefore can also be considered a metric for real time guidance of the drug release. (FIGS. 8a and 8b).

Unlike cancer cells, the identically PNB- and drug-treated normal cells demonstrated a much better survival: 83% for L-Dox-C225 treated cells (FIG. 8a) and 62% for M-Ptx-C225 treated cells (FIG. 8b). Thus, cancer cell-specific PNBs provided a high selectivity of the drug release that was not activated in normal cells. Drugs administered alone required an 18-fold higher concentration of doxorubicin (85 μg mL⁻¹) and a 15-fold higher concentration of paclitaxel (1 μg mL⁻¹) to bring the complex viability of cancer cells to the above low levels achieved with the PNB-enhanced release. These high drug doses also reduced the complex viability of normal cells by 3 to 5-fold demonstrating high non-specific toxicity of both drugs at therapeutic doses. Therefore, the PNB release mechanism overcame the drug resistance of cancer cells and spared normal cells by not releasing drugs from non-specifically internalized nanocarriers and by radically reducing drug doses. The PNBs were equally efficient at releasing two principally different drugs, soluble doxorubicin and poorly soluble paclitaxel from two principally different nanocarriers, liposomes and micelles, respectively.

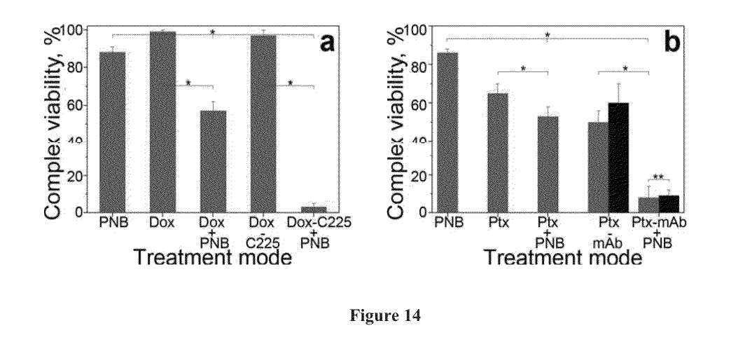

Influence of the targeting factors on PNB-induced drug release. The PNB release mechanism relies upon targeting and co-localization of drug-loaded nanocarriers and gold NPs. We used EGFR-positive HN31 (cancer) cells to estimate the importance of such co-localization by comparing the effect of plain (non-conjugated) and C225-conjugated liposomes and micelles. Plain nanocarriers increased the cancer cell viability by several-fold both for doxorubicin (FIG. 14a) and for paclitaxel (FIG. 14b) compared with the conjugated carriers. The non-specific uptake of plain nanocarriers apparently prevented their efficient mixing with gold NPs through receptor-mediated endocytosis. The high sensitivity of the PNB release mechanism to the co-localization of nanocarriers and GNPs can be explained by the localized nature of the PNB impact. Next, we used two different molecular targets in cancer cells (instead of one, EGFR, in the previous experiments), and targeted gold GNP-C225 to EGFR and paclitaxel to nucleosomes by conjugating them to a 2C5 antibody we previously synthesized. The 2C5 antibody can be synthesized according to the method disclosed in Iakoubov, L., Rokhlin, O. & Torchilin, V. “Anti-nuclear autoantibodies of the aged reactive against the surface of tumor but not normal cells,” Immunol. Lett. 47, 147-149 (1995). The effect of such “dual” targeting was similar to that observed for a single target, EGFR (FIG. 14b). Therefore, the intracellular co-localization of nanocarriers and gold NPs can also be achieved by using one or several different molecular targets and matching vectors.

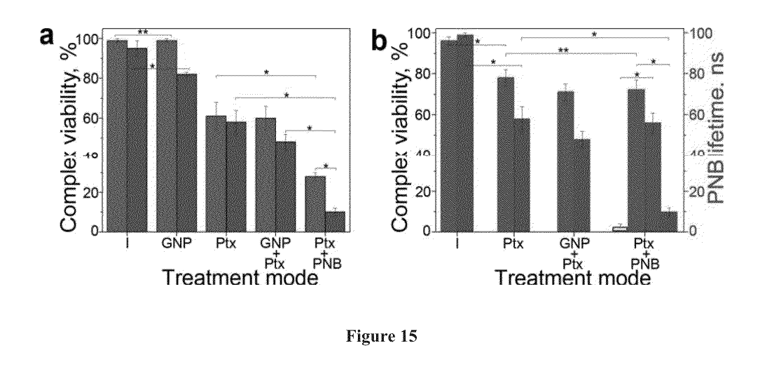

Short-term therapeutic response to quadrapeutics and other treatments in vitro. After pre-treating both cancer and normal cells with several combinations (FIGS. 15a-b) including Ptx-C225 at a further reduced dose of Paclitaxel of 0.05 μg mL⁻¹, GNP-C225 and single laser pulses (532 nm, 70 ps, 40 mJ cm⁻²), we exposed the same cells to a single dose of X-rays (10 Gy). The radiation treatment was administered within 60 min after PNB generation, i.e. when the intracellular concentration of the released drug was close to the maximal. The concomitant application of GNP-C225, Ptx-C225 and X-rays further reduced the viability of cancer cells to 48±4% (FIG. 15a, “GNP+Ptx” mode), thus confirming the well-known radio-sensitizing effect of the drug. However, in all the above cases, the gains in cancer cell destruction were rather incremental and much lower than that achieved previously with the PNB-enhanced drug release without X-rays.

In contrast, when the same X-ray dose was applied within 30 minutes after PNB generation in cancer cells pre-treated with Ptx-C225 and GNP-C225 (i.e. when the local intracellular concentration of the released drug was expected to be the maximal), we observed an almost four-fold reduction in the cancer cell viability down to 10±2% (FIG. 15a, “Ptx+PNB” mode) compared to the effect of the same drug and X-rays alone (FIG. 15a, “Ptx” mode). The PNB lifetimes were 55-60 ns in cancer cells and close to zero in normal cells (FIG. 15b). Thus, the “PNB-drug-radiation” mode provided the maximal destruction of cancer cells. The viability of normal cells after identical treatment with GNPs, the encapsulated drug, single laser pulses and X-rays remained relatively high 71±5% (FIG. 15b), thus showing the high selectivity and low non-specific toxicity of this combination. We attribute the observed increase of the efficacy and selectivity of cancer cell destruction to the intracellular co-localization of the released drug and the amplified x-rays (FIG. 1c). Both effects were associated with cancer cell-specific formation of the largest mixed clusters of drug-loaded nanocarriers and gold NPs that generated PNBs and amplified the external x-rays. Although this experiment did not aim to optimize the radio-sensitivity of cancer cells and to measure long-term effects, it shows that plasmonic nanobubbles and nanoclusters can selectively enhance two standard therapeutics in cancer cells to overcome their resistance to therapies and to reduce non-specific toxicity.

Therapeutic Responses to Drug Nanocarriers, GNPs, Laser Pulses and X-Rays

Evaluation of the long-term therapeutic response with a clonogenic test. We used EGFR-positive HN31 cells and EGFR-negative NOM9 cells. Drug carriers and GNP-C225 conjugates (2.4×10¹⁰ GNPs mL⁻¹) were separately administered to the cells (for 24 hours with Doxil-C225 (2 μg mL⁻¹) and 4 hours with micellar Paclitaxel (33 ng mL⁻¹)) and were then washed off prior to laser treatment. Thus the cells were exposed only to the internalized drug during the follow-up generation of PNBs. One to six hours afterwards, a single dose of X-rays (0-25 Gy) was applied. To evaluate the therapeutic efficacy of standard chemotherapy, unconjugated Doxil (0-80 μg mL⁻¹) and micellar Paclitaxel (0-1 μg mL⁻¹) were applied.

To evaluate their colony-forming ability, the cells were trypsinized and replaced in 100 mm dishes in a free medium immediately after the treatment of samples. After 10 days of cultivation, the cells were stained with 0.5% crystal violet in absolute ethanol, and colonies with more than 50 cells were counted under a microscope. Plating efficiency was defined as the percentage of cells seeded that grew into colonies under a specific culture condition of a given cell line. The survival fraction, expressed as a function of different treatment conditions as described previously by Franken N. et al. (Franken, N. A. P., Rodermond, H. M., Stap, J., Haveman, J. & van Bree, C. Clonogenic assay of cells in vitro. Nat. Protocols. 1, 2315-2319 (2006)). All experiments were performed three times.

The long-term response was analyzed for clonogenicity and the short-term response was assayed for necrosis for both cell lines. Two established anti-cancer drugs were evaluated. Water-soluble doxorubicin was encapsulated into PEG-coated 90 nm Doxil liposomes and insoluble paclitaxel was loaded into 14 nm micelles. The chemotherapy mode (FIG. 4a) used each encapsulated drug alone and demonstrated the high drug resistance of HNSCC cells and non-specific toxicity at therapeutic doses.

Next, drug nanocarriers and GNPs, each conjugated with a C225 antibody, were simultaneously administered to cancer and normal cells. Drug doses were radically reduced to lower the non-specific toxicity associated with the non-specific uptake and non-triggered release of drug in normal cells. After incubation, free GNPs and nanocarriers were washed off cells. Doxil, applied at a 40-fold reduced dose of 2 μg mL⁻¹ (compared to a clinical dose), resulted in a good survival of both cancer and normal cells (FIGS. 4d, 4e and 8a). Micellar paclitaxel, applied at a 30-fold reduced dose of 0.033 μg mL⁻¹ (compared to reported in vivo doses), also resulted in good survival of both cell lines (FIGS. 4d, 4e and 8b). At this stage, the problem of non-specific toxicity was resolved but the therapeutic efficacy remained low.

The exposure of GNP-treated cells (without drug nanocarriers) to a single near-infrared laser pulse (70 ps, 780 nm) at a low fluence of 45 mJ cm⁻², did not reduce cell clonogenicity (FIG. 4b) or short-term viability (FIG. 8). However, with an increase in the laser fluence, we observed an increase in the PNB lifetime only in the cancer cells, but not in normal cells (FIG. 13). The increased fluence, in turn, led to a gradual decrease in clonogenicity among cancer cells but not among normal cells (FIG. 4b). The lethal effect of large PNBs (lifetime>200 ns) was studied previously in detail. It was not employed in this work, in which we used only small PNBs with lifetimes below 80 ns and a low and safe laser fluence of 45 mJ cm⁻² (FIG. 4b).

In contrast, the co-localization of such small non-invasive PNBs with pre-administered drug nanocarriers in cancer cells significantly reduced their clonogenicity (FIG. 4d, mode “D+PNB” vs “PNB”) and caused a nearly complete loss of their short-term viability (FIG. 8, modes Dox+PNB and Ptx+PNB vs PNB) compared to drugs (FIG. 4a) and PNBs (FIG. 4b) administered separately. Unlike cancer cells, the identically PNB- and drug nanocarrier-treated normal cells demonstrated a much greater clonogenicity (FIGS. 4e and 8) that was similar to that for intact normal cells. Thus, PNB-induced intracellular release of two principally different drugs, soluble doxorubicin and insoluble paclitaxel from two principally different nanocarriers, liposomes and micelles, demonstrated the enhanced and cancer cell-specific therapeutic effect. At the same time, the non-triggered slow diffusive leak of these drugs from non-specifically internalized nanocarriers was safe for normal cells due to the 30-40 fold reduction in drug dose (FIGS. 4e and 8).

The therapeutic efficacy the PNB-induced release depended upon the intracellular co-localization of the drug nanocarriers and PNBs. This was achieved by the co-targeting of drug nanocarriers and GNPs that led to the formation of mixed GNP-drug nanocarrier clusters. Without such co-targeting, the localized mechanical impact of PNBs apparently did not reach the otherwise randomly internalized drug nanocarriers, resulting in a relatively poor therapeutic effect (FIG. 14, the modes Dox+PNB and Ptx+PNB). The efficient intracellular co-localization of GNPs and drug nanocarriers was achieved by targeting one (EGFR, FIG. 14a, the mode Dox-C225+PNB) or two different molecular targets (EGFR for GNPs and nucleosomes for micellar paclitaxel, FIG. 14, the mode Ptx-mAb+PNB, black).

X-rays alone without drugs and GNPs were efficient only at high doses due to the resistant nature of HN31, but such doses also caused high non-specific toxicity (FIG. 4c). To minimize the non-specific toxicity, the X-ray dose was reduced to 4 Gy (by 15-fold compared to the clinical dose of 60-70 Gy for HNSCC) and was applied in a single treatment. The effect of GNP clusters alone improved the therapeutic efficacy of X-rays in cancer cells incrementally compared to X-rays alone (FIG. 4d, the mode GNP+XR and FIG. 15a, the mode GNP). This enhancement can be attributed to the intracellular “amplification” effect of GNP clusters that emit secondary electrons. The pre-treatment of cancer cells with small PNBs prior to the application of X-rays did not improve the therapeutic effect of X-rays (FIG. 4d, the mode PNB+XR1 versus GNP+XR).

Next, modeling of a standard chemoradiation therapy by incubating the cells with drug nanocarriers alone (without generating PNBs) and the follow-up exposure to X-rays resulted in a predictable suppression of the clonogenicity (FIG. 4d, the mode D+XR1) and short-term viability (FIG. 15a, the mode Ptx) of cancer cells, due to well-known synergy of drugs and X-rays. However, the applied low doses of drugs and X-rays while being safe for normal cells (FIG. 4e, the mode D+XR1) did not prevent the growth of cancer cells (FIG. 4d, the mode D+XR1).

In contrast, the administration of all four modalities that combined PNB-enhanced drug release and GNP-amplified X-rays in a quadrapeutic procedure resulted in a strong, cancer cell-specific suppression of clonogenicity (FIG. 4d, the mode D+PNB+XR1) and short-term loss of viability (FIG. 15a, the mode Ptx+PNB, purple), and spared normal cells (FIG. 4e, the mode D+PNB+XR1, FIG. 15b, the mode Ptx+PNB). This strong and cancer cell-specific therapeutic effect resulted from a single quadrapeutic sequence: GNPs and drugs—laser pulses (PNBs)—X-rays. X-rays were administered within one hour of PNB generation. An even stronger therapeutic effect was achieved, however, after X-rays were applied in 6 hours after the PNB treatment (FIG. 4d, the mode D-PNB-XR6). In this case, we observed complete termination of cancer cell growth with Doxil (Dox at 2 μg mL⁻¹+GNP at 2.4×10¹⁰ GNPs mL⁻¹+laser pulse at 45 mJ cm⁻²+X-rays at 4 Gy) as well as a strong therapeutic effect for paclitaxel (Ptx, 33 ng mL⁻¹+GNP, 2.4×10¹⁰ GNPs mL⁻¹+laser pulse, 45 mJ cm⁻²+X-rays, 4 Gy). In this mode, the clonogenicity of cancer cells was reduced by more than two orders of magnitude compared to chemoradiation therapy with Doxil (FIG. 4f). The effect of the delayed X-rays can be attributed to the well-known radio-sensitization of cancer cells by drugs. Identically treated normal cells demonstrated the high long—(FIG. 4f) and short-term (FIG. 15b, green) viability close to that of intact cells. These experiments reveal the unique combination of a radical increase in therapeutic efficacy with a reduction in non-specific toxicity associated with the 30 to 40 fold reduction in drug doses and the 15 fold reduction in X-ray doses. This was achieved through the intracellular mechanical impact of PNBs that provided a true single cell level rapid treatment and increased the efficacy of both drugs and X-rays.

The Evaluation of Quadrapeutics in Animal Models of Head and Neck Squamous Cell Carcinoma (HNSCC).

We next studied the basic mechanisms of quadrapeutics in vivo, and then evaluated its efficacy in dealing with one of the major challenges of HNSCC microscopic residual disease (MRD) owing to the incomplete surgical resection of a tumor and the high resistance of HNSCC to chemo- and chemoradiation therapies.

We first analyzed the efficacy of the systemic delivery of GNPs, and of PNB generation and detection in a primary xenograft murine model using the same HN31 cell line used in in vitro studies. The systemic administration of the 60 nm GNP-C225 conjugates (0.8 μg g⁻¹) resulted in large GNP clusters only in tumors (FIG. 16a), while the normal adjacent tissue showed only single unclustered GNPs (FIG. 16b) showed by TEM imaging of tumor and normal adjacent tissue slices 24 hours after GNP administration. Quantitative analysis demonstrated a high cluster size contrast for tumor versus normal tissue (FIG. 16c), which was similar to that previously observed in vitro (FIG. 12a). Exposure of a tumor to a single broad near-infrared laser pulse (780 nm, 45 mJ cm⁻²) resulted in PNBs detected in vivo via acoustic time-responses (FIGS. 6a and 17a) whose amplitude was used as a metric of a PNB. Similarly to in vitro experiments (FIG. 13), we found that in vivo PNBs had high tumor specificity, sensitivity (which can be increased with the laser fluence) (FIG. 17b) and spectral selectivity in the near-infrared region for colloidal GNPs (FIG. 17c). Optical tissue penetration depth in the near-infrared is up to 10 mm. Such good near-infrared performance of GNPs that are normally considered to be “useless” in the near-infrared region was achieved through our novel method of non-stationary optical excitation of GNPs with picosecond pulses.

Next, we evaluated the quadrapeutics against primary HNSCC tumors in two murine xenograft models. In the first model, we injected a low number of the pre-treated HN31 cells s.c. into the mouse flank and monitored the tumor growth (FIGS. 5a-c). We compared four groups of animals with: intact HN31 cells, cells treated with 55 ns PNBs (without drugs or X-rays), cells treated with Doxil (2 μg mL⁻¹) and X-rays (4 Gy) or cells treated with quadrapeutics (Doxil-C225, 2 μg mL⁻¹, GNP-C225, 2.4×10¹⁰ GNP mL⁻¹, laser pulse, 780 nm, 45 mJ cm⁻² at 24 hours after GNP and drug administration, and X-rays, 4 Gy, 6 hours after laser treatment). Tumors were characterized by their volume and the rate of incidence. After 15 days, tumors in the animals injected with intact cells reached the maximal size allowed (FIG. 5a, left flank). Similarly, large tumors were observed after PNB treatment alone (FIG. 5a, right flank). A standard chemoradiation therapy also did not prevent tumor growth (FIG. 5b, left flank). In contrast, either no or very small tumors were found in the quadrapeutic group (FIG. 5b, right flank), with more than ten-fold reduction in tumor volume compared to that in chemoradiation-treated group (FIG. 5c).

In the second model, we used Luciferase-encoded HN31 cells for primary HNSCC xenograft tumors. The therapeutic response to a single treatment in vivo was compared among four animal groups. Quadrapeutic group received GNP-C225 (0.8 μg g⁻¹) and Doxil-C225 (1 mg kg⁻¹) systemically. After 24 hours, tumor areas were scanned with broad near-infrared laser pulses (780 nm, 45 mJ cm⁻²) and then after 6 hours were exposed to X-rays (4 Gy). Generation of PNBs in tumors was confirmed via their acoustic signals. Chemoradiation group received same doses of drug and X-rays. PNB group received same doses of GNPs and laser pulses, and the control group received no treatment. After a single treatment, all animals were monitored weekly for the tumor volumes and bioluminescence (FIGS. 5d-g). A quadrapeutic treatment suppressed tumor growth after the first week (FIGS. 5e and 5g) by more than 30-fold compared to untreated tumors and by 17-fold compared to chemoradiation therapy (FIG. 5f). Small PNBs (GNP and laser-treated group) or chemoradiation (drug and X-ray—treated group) did not prevent the tumor growth and demonstrated relatively low therapeutic efficacy (FIG. 5g). In contrast, quadrapeutics radically accelerated and enhanced the treatment (FIG. 5g). This clearly proved a synergistic nature of the quadrapeutic mechanism that employed all four therapeutic components. Although the post-effect of a single quadrapeutic treatment gradually decreases during the second and third weeks, it still remains 4-6 folds higher than that of chemoradiation (FIG. 5g). In this work, we intentionally used a single treatment model in order to compare the standard and quadrapeutic approaches under identical conditions.

Next, we evaluated the translational potential of quadrapeutics for an intra-operative diagnosis and adjuvant treatment of MRD, a highly aggressive and lethal cancer which represent one of the major challenges in HNSCC treatment. Xenograft HNSCC tumor was grown from GFP-encoded cell. GNP and Doxil conjugates were administered systemically as described above, 24 hours prior to the tumor resection. Immediately after the full resection of the tumor, the surgical margins were scanned with a broad near-infrared pulsed laser beam (780 nm, 45 mJ cm⁻²). PNBs were detected acoustically during the laser scan by using an ultrasound sensor (FIGS. 6a and 17a). PNB acoustical responses were obtained for a primary tumor, surgical margins, and adjacent muscle tissues of GNP-treated and intact animals, and their amplitudes were analyzed as tumor metrics (FIG. 6b). The total time needed for PNB generation and detection in the surgical margins was less than 30 seconds. The detected in surgical margins PNBs indicated the presence of the MRD (FIGS. 6b and 17d). It should be noted that a standard photoacoustic system applied post-operatively to the same animals at the same laser wavelength failed to detect MRD (FIG. 17d). This experiment demonstrated the high cancer sensitivity, specificity and speed of the PNB diagnosis of MRD in a biopsy-free and real-time manner.

The intra-operative adjuvant treatment of MRD was analyzed for three groups: surgery, surgery with chemoradiation (Doxil, 1 mg kg⁻¹, 24 hours before surgery and X-rays, 4 Gy, 6 hours after surgery) and surgery with the quadrapeutics (Doxil, 1 mg kg⁻¹ concomitantly with GNPs (0.8 μg g⁻¹), 24 hours before surgery, laser scan during the surgery (780 nm, 45 mJ cm⁻²) and X-rays, 4 Gy, 6 hours after surgery). The presence of MRD was confirmed intra-operatively via the PNB acoustic signals immediately after the tumor resection (FIGS. 17a and 17d). The local recurrence of HNSCC after the treatment was monitored in animals by visual observation and imaging of HNSCC fluorescence (HN31 cells were encoded with Green Fluorescent Protein) (FIGS. 6c and 6d). Surgery alone resulted in tumor recurrence in 2-4 weeks in 100% of animals (FIGS. 6c and 6f). Surgery and adjuvant chemoradiation also failed to prevent tumor recurrence (FIGS. 6d and 6f). In contrast, surgery and quadrapeutics led to much smaller or no recurrent tumors in animals with barely detectable fluorescent HNSCC-specific signals and a three-fold lower incidence of tumor recurrence after 28 days as compared to chemoradiation therapy (FIGS. 6e and 6f). Thus, PNBs and quadrapeutics provided an efficient intra-operative detection and elimination of MRD in a single theranostic procedure.

Comparison of Quadrapeutics with Current Approaches.

Quadrapeutics consists of three important innovations, each of which makes it more effective than current cancer treatment modalities. These three innovations are detailed below, and a side-by-side comparison of current approaches with quadrapeutics is presented in Table 1 below.

TABLE 1
Side-by-side comparison of current approaches with quadrapeutics.

| Current Method | Limitation | | Quadrapeutics Solution |
|---|---|---|---|
| Drug delivery | 1. | Low release efficacy due to slow diffuse release | Radically enhanced efficacy (>3 fold) |
| with various | | of the drug (>10 min) | due to high speed of intracellular drug |
| nanoparticles | | | release |
| | 2. | No on-demand release | On-demand release within nanoseconds |
| | | | due to explosive localized disruption of |
| | | | nanocarriers |
| | 3. | High dose of the drug | 90-98% reduction in a drug dose |
| | 4. | High non-specific toxicity due to uptake of | Low non-specific toxicity due to high |
| | | nanoparticles by normal cells/tissues | cancer cell selectivity of PNBs |
| | 5. | Long treatment time | Short single laser pulse treatment |
| Drug delivery | 1. | Low selectivity of the drug release due to uptake | High selectivity of the drug release due |
| and therapy | | of nanoparticles by normal tissues and a de- | to high cancer cells selectivity of PNBs |
| with: | | localized release mechanism | |
| External | 2. | Complex and unstable nanocarriers | Simple, safe clinically-validated one- |
| energy GNPs | | | component GNPs and drug nanocarriers |
| Theranostic | | | self-assembled by cancer cells into |
| nanoparticles | | | mixed clusters |
| | 3. | High energy (>1 J/cm²) | Low energy (<50 mJ/cm²) |
| | 4. | Prolonged exposure time (>1 min) | Single laser pulse treatment (<1 second) |
| | 5. | High non-specific toxicity | Low non-specific toxicity |
| Laser micro- | 1. | High energy due to the bulk photothermal | Low energy due to intracellular PNB |
| surgery and | | mechanisms | mechanism |
| thermal therapy | 2. | Therapeutic selectivity depends upon laser beam | Single cancer cell selectivity does not |
| | | pointing and size | depend on laser beam pointing accuracy |
| | | | or size |
| | 3. | May not prevent recurrence of HNSCC | Will prevent recurrence of HNSCC |
| GNP-mediated | 1. | Low selectivity within a laser aperture due to | Single cancer cell selectivity does not |
| thermal therapy | | thermal diffusion | depend upon laser beam size, no |
| | | | thermal impact |
| | 2. | High dose and exposure time | Low dose and single pulse exposure |
| | 3. | High non-specific toxicity | Low non-specific toxicity |
| | 4. | Limited efficacy | High efficacy of explosive, non-thermal |
| | | | mechanism |
| GNP-enhanced | 1. | Low therapeutic gain (<2-fold) | High therapeutic gain (10-100-fold) |
| radiotherapy | 2. | High GNP dose | Reduced to 0.01% GNP dose |
| | 3. | Low selectivity of external X-rays and non- | High selectivity and gain of X-ray |
| | | specific uptake of GNPs by normal cells and | amplification due to cancer cell-specific |
| | | tissues | large GNP clusters |

Intracellular synergy of several novel mechanisms of high efficacy and selectivity of quadrapeutics employs four modalities. Drug nanocarriers and GNPs are conjugated to cancer-specific antibodies and are administered separately, either locally or systemically. Rather than protecting targeted cancer cells against any external impact, receptor-mediated endocytosis creates a novel therapeutic structure of a large mixed intracellular cluster of GNPs and drug nanocarriers (FIG. 1a). Separate administration of standard GNPs and drugs eliminates the need to engineer and approve new therapeutic complexes of drugs and GNPs.

Next, the surgical bed or tumor area is exposed to a single, short, near-infrared laser pulse that provides a tissue penetration depth of up to 10 mm when administered transdermally or more than 100 mm using standard clinical laser catheters and guides). GNPs absorb the optical pulse and instantaneously evaporate the nearby medium, thus producing an expanding and collapsing vapor nanobubble, a process we recently invented and termed a plasmonic nanobubble (PNB). It is important to note that a PNB is not a particle but is instead a transient event lasting mere nanoseconds. A PNB delivers a highly localized, mechanical, non-thermal impact on drug nanocarriers and cellular endosomes. This PNB “nano-explosive” effect, co-localized with nanocarriers, ejects the drug from the endosome into the nano-volume of cytoplasm in nanoseconds, instantaneously creating high concentrations of the drug in cancer cells only (FIGS. 1b, 2 and 3). This on-demand drug release employs small PNBs (0.1-0.5 μm in diameter and a lifetime<70 ns).

Another benefit of the PNB is that, unlike in other therapeutic and surgical approaches, its therapeutic effect does not use heat. Furthermore, a PNB effectively insulates the cell from laser-heated GNPs owing to the very low thermal conductivity of the vapor, meaning that the bulk temperature of a cell remains at the ambient level. This property also efficiently reduces the laser pulse dose needed for quadrapeutics to three to six orders of magnitude less than required by other nanoparticle-based drug delivery and therapeutic methods (Table 1).

Importantly, the laser dose range with quadrapeutics (10-40 mJ cm⁻²) is within the American National Standards Institute safety limits. These unique properties principally differentiate PNBs from all other cellular agents, including metal nanoparticles. The fourth modality, X-rays, are locally amplified in cancer cells by the same GNP-nanocarrier cluster (FIG. 1c). However, the most powerful therapeutic effect of quadrapeutics is achieved through the intracellular interaction and synergy of the released drug with the amplified X-rays. Based on our data (FIGS. 4 and 5), the synergy of the four quadrapeutic modalities enhances the efficacy of chemoradiation therapy in HNSCC cells up to 100-fold, reduces the dosages of drugs and X-rays by 90-98%, and shortens the treatment time to a single, short procedure. All of which are benefits that cannot be provided by any of the current materials and technologies (Table 1).

Rapid highly sensitive detection of micro-tumors. Another crucial benefit of a PNB is that it emits a strong pressure pulse that can be detected instantaneously, thus enabling the intraoperative diagnosis of microscopic residual disease with high sensitivity and specificity via scanning of the surgical margins (FIG. 5a) and ensuring immediate and precise feedback on them. Current intra-operative diagnosis of microscopic residual disease, which includes collection and histological analysis of biopsy samples (frozen sections), is slow (20 minutes to 3 days) and often inaccurate. As a result, when using these current methods, it is nearly impossible to detect micro-tumors in real time.

In contrast, however, PNBs can be detected instantaneously, thereby eliminating the need for biopsy. While the PNB method is technically similar to photo-acoustic imaging, the GNPs used in the photo-acoustic method do not emit pulses that are as strong as those emitted by PNBs. In fact, when tested in an animal model of HNSCC, the photo-acoustic method failed to detect microscopic residual disease due to its low sensitivity, whereas the PNB method did (FIG. 17d).

Secondly, since PNBs are generated only in cancer cells—not in normal cells—the PNB method makes it possible to increase the specificity of diagnosis (see below). Thus, the PNB method is more sensitive, specific, and straightforward than GNP-based photo-acoustic methods. And, finally, PNBs are also advantageous over intra-operative optical diagnostic methods, which cannot provide the high level of cancer specificity offered by PNBs. Consequently, PNBs can guide surgery better than current methods (including MRI), which are both more expensive and less accurate.

The high cancer cell specificity of PNB-supported diagnosis and therapy. It is based on the unique cluster-threshold mechanism of PNBs. Firstly, unlike other photo-induced biomedical modalities such as heat, light, or sound, PNBs have a threshold nature: PNBs do not emerge if the fluence (energy per square centimeter) of the laser pulse is below the PNB threshold. Secondly, we discovered a strong dependence of the PNB threshold upon the GNP cluster size. Large clusters, which are self-assembled by cancer cells (FIGS. 12 and 16) have a low PNB threshold, whereas small clusters and single GNPs in normal cells have a high PNB threshold (FIGS. 12b, 13a, and 17b). The unavoidable non-specific uptake of GNPs by normal cells results in a low number of internalized GNPs, usually single GNPs or small clusters (FIGS. 12a and 16). When cancer and normal cells are exposed to a laser pulse of a low enough fluence which is insufficient to generate PNBs around single GNPs in normal cells, the PNBs are generated only in cancer cells, as we have verified both in vitro and in vivo (FIGS. 13 and 17). Unlike with other nanoparticles, the cluster-threshold mechanism of PNB generation increases cancer cell selectivity for PNBs by more than one order of magnitude. The non-specific uptake of drug nanocarriers and their non-triggered slow diffuse leakage in normal cells does not increase the non-specific toxicity (FIGS. 4 and 15) because quadrapeutics employs very low drug doses (90-98% lower than current clinical practices).

Therefore, quadrapeutics has numerous benefits over a range of current approaches to treatment of resistant cancers. The combination of therapeutic efficacy, selectivity and speed, and highly sensitive real-time diagnosis provided by quadrapeutics, cannot be matched by any currently used methods (Table 1). The major strategic innovation of quadrapeutics is in the transformation of chemoradiation therapy into an on-demand precise cell-level modality for intra-operative, adjuvant, and preventive therapy for HNSCC and other cancer types.

What is claimed is:

1. A method comprising:
introducing into a cell at least one gold nanoparticle and separately at least one therapeutic agent; and
applying to the cell an optical pulse sufficient to produce a nanobubble.

2. The method of claim 1, wherein the therapeutic agent is encapsulated in a carrier.

3. The method of claim 1, wherein the gold nanoparticle and the therapeutic agent are functionalized with a targeting agent.

4. The method of claim 1, wherein the gold nanoparticle and the therapeutic agent are functionalized with a targeting agent chosen from one or more of an antibody, an aptamer, and a peptide.

5. The method of claim 1, wherein the at least one gold nanoparticle and the at least one therapeutic agent form a cluster in the cell.

6. The method of claim 1, wherein the cell is a cancer cell.

7. The method of claim 1, wherein the therapeutic agent is a cytostatic or cytotoxic drug, a genetically-active material or a signal-activating material.

8. The method of claim 7, wherein the cytostatic or cytotoxic drug is cisplatin, doxorubicin, paclitaxel, or 5-furourocil.

9. The method of claim 1, further comprising applying a dose of radiation to the cell.

10. The method of claim 1, further comprising detecting acoustic responses.

11. A composition comprising at least one gold nanoparticle separate from and disposed adjacent to at least one therapeutic agent.

12. The composition of claim 11, further comprising a nanobubble disposed around the at least one gold nanoparticle.

13. A system comprising:
a composition comprising at least one gold nanoparticle separate from and disposed adjacent to at least one therapeutic agent; and
a laser disposed operable to the composition, the laser is capable of generating an optical pulse sufficient to create a nanobubble around the composition.

14. The system of claim 13, wherein the therapeutic agent is encapsulated in a carrier.

15. The system of claim 13, wherein the gold nanoparticle and the therapeutic agent are functionalized with a targeting agent.

16. The system of claim 13, wherein the gold nanoparticle and the therapeutic agent are functionalized with a targeting agent chosen from one or more of an antibody, an aptamer, and a peptide.

17. The method of claim 13, wherein the at least one gold nanoparticle and the at least one therapeutic agent form a cluster.

18. The system of claim 13, further comprising a detector.

19. The system of claim 13, further comprising a radiation source.
